(19) United States
(12) Patent Application Publication
(10) Pub. No.: US 2008/0199855 A1
(43) Pub. Date: Aug. 21, 2008

(54) Identification And Characterization Of A Subset of Glioblastomas Sensitive To Treatment With Imatinib

(76) Inventors: Monica Nister, Stockholm (SE); Daniel Hagerstrand, Stockholm (SE); Goran Hesselager, Uppsala (SE); Arne Ostman, Stockholm (SE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/568,781

(22) PCT Filed: May 13, 2005
(86) PCT No.: PCT/EP05/05269

§ 371 Date: Oct. 10, 2007

(30) Foreign Application Priority Data
May 14, 2004 (GB) 0410883.3
Nov. 16, 2004 (GB) 0425257.3

(51) Int. Cl. C12Q 1/68; C40B 40/06

(52) U.S. Cl. 435/6; 506/16

(57) ABSTRACT

The present invention relates to methods for in vitro diagnosing a cell proliferative disease in a mammal, for predicting the behaviour of a mammal having a cell proliferative disease in response to a medical treatment using at least one PDGF receptor antagonist, and for selecting a mammal having a cell proliferative disease and predicted to be responsive to a medical treatment using at least one PDGF receptor antagonist, by using given genetic markers.

The present invention relates to methods for in vitro diagnosing a cell proliferative disease in a mammal, for predicting the behaviour of a mammal having a cell proliferative disease in response to a medical treatment using at least one platelet-derived growth factor (PDGF) receptor antagonist, and for selecting a mammal having a cell proliferative disease and predicted to be responsive to a medical treatment using at least one PDGF receptor antagonist, by using given genetic markers.

Glial tumors are according to World Health Organization standards graded into four grades. Grading is based on histological criteria such as nuclear atypia, mitotic activity, vascular thrombosis, micro vascular proliferation and necrosis. Grade II tumors are generally divided into astrocytomas, oligodendrogliomas and mixed oligoastrocytomas, depending on cell type origion. Grade III is divided into anaplastic astrocytomas and anaplastic oligodendrogliomas. Grade IV, the highest form is commonly known as glioblastoma multiforme (GBM).

Glioblastoma (GBM) is thus the most common malignant brain tumor of the adult. Treatment is presently based on surgery, radiation therapy and chemotherapy. However, with these treatment modalities, responses are extremely poor. Two-year survival for GBM patients is less than 7.5% (Maher et al., 2001). Identification of novel treatment strategies is therefore highly warranted.

Based on the clinical course of the disease, and characterization of the genetic alterations, GBM has been broadly divided into primary and secondary GBMs (reviewed in Maher et al., 2002). Primary GBMs are associated with amplification of a mutationally altered EGF receptor, whereas secondary GBMs are characterized by p53 mutations and overexpression of PDGF and PDGF receptors. As compared to primary GBMs, secondary GBMs occur in younger patients. Recent studies have also identified a novel subset among the secondary GBMs characterized by over-expression of genes on chromosome 12q13-14 (Mischel et al., 2003).

The combined expression of PDGF and PDGF receptors in a subset of GBMs is compatible with a functional role of autocrine PDGF receptor signaling in GBM growth. This notion has been supported by experimental approaches. Firstly, GBM-like tumors can be induced in mice after overproduction of PDGF in mice brain (Dai et al., 2001; Uhrbom et al., 1998). Secondly, experimental therapy studies with different types of PDGF receptor inhibitors have demonstrated that growth of GBM derived cell lines can be blocked by interference with PDGF receptor signaling (Kilic et al., 2000; Shamah et al., 1993; Strawn et al., 1994).

The availability of clinically useful PDGF receptor antagonists, like compound I, has demonstrated the possibility to obtain therapeutic effects by interfering with PDGF receptor signaling in tumors (reviewed in Pietras et al., 2003). Compound I is an orally available tyrosine kinase inhibitor which, in addition to PDGF receptors, also blocks the tyrosine kinase activity of c-Kit, c-Abl, Bcr-Abl and Arg (reviewed in Capdeville et al., 2002). The clinical utility of compound I has been well demonstrated in studies on patients with CML and GIST, which are associated with aberration of Bcr-Abl and c-Kit, respectively (Demetri et al., 2002; O'Brien et al., 2003).

As mentioned above, since no satisfying treatment of GBM does exist to date, there is a need for finding new therapeutic strategies for successfully treating mammals, preferably humans, afflicted by GBMs and, more generally, by cell proliferative diseases.

As used herein, a “mammal” is a warm-blooded mammal, including human.

A “biological sample” is, according to the invention, a sample of a mammal obtained from any biological material separated from the mammalian body, including tissue, cell, plasma, serum, cell or tissue lysate, and preferably tumor tissue. Such a sample may be obtained by, e.g., a biopsy.

The expression “platelet-derived growth factor (PDGF) receptor antagonist” herein refers to any agent which blocks PDGF receptor signaling, including, e.g., antibodies targeting PDGF ligands or receptors, recombinant forms of soluble receptors or aptamers preventing PDGF binding to receptor, as well as LMW compounds directly interfering with PDGF receptor kinase activity such as compound I (see below) and other agents with similar mechanism of action, as well as pharmaceutically acceptable salts thereof. Preferably, a PDGF receptor antagonist useful for operating the present invention is compound I below, or a pharmaceutically acceptable salt thereof.

The expression “pharmaceutically acceptable” means that which is useful in preparing a pharmaceutical composition that is generally safe, non-toxic, and neither biologically nor otherwise undesirable and includes that which is acceptable for mammal, preferably human, pharmaceutical use.

A <<pharmaceutically acceptable salt >> is intended to mean a salt that retains the biological effectiveness of the free acids and bases of a specified compound (e.g., compound I or other PDGF receptor antagonists) and that is not biologically or otherwise undesirable. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

A desired salt may be prepared by any suitable method known in the art, including treatment of the free base of a PDGF receptor antagonist such as compound I with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid or ethanesulfonic acid, or the like.

In the case of compounds, salts, or solvates that are solids, it is understood by those skilled in the art that the compounds, salts, and solvates may exist in different crystal forms, all of which are intended to be within the scope of the present invention and specified formula.

A “pharmaceutical composition” is also referred to herein by the synonymous terms “pharmaceutical preparation” or “drug”.

PDGF receptor antagonists, including compound I, and pharmaceutically acceptable salts or solvates thereof, may be administered as pharmaceutical compositions in any pharmaceutical form recognizable to the skilled artisan as being suitable. Suitable pharmaceutical forms include solid, semisolid, liquid, or lyophilized formulations, such as tablets, powders, capsules, suppositories, suspensions, liposomes, and aerosols. Pharmaceutical compositions may also include suitable excipients, diluents, vehicles, and carriers, as well as other pharmaceutically active agents, depending upon the intended use or mode of administration.

Acceptable methods for preparing suitable pharmaceutical forms of the pharmaceutical compositions may be routinely determined by those skilled in the art. For example, pharmaceutical preparations may be prepared following conventional techniques of the pharmaceutical chemist involving steps such as mixing, granulating, and compressing when necessary for tablet forms, or mixing, filling, and dissolving the ingredients as appropriate, to give the desired products for oral, parenteral, topical, intravaginal, intranasal, intrabronchial, intraocular, intraaural, and/or rectal administration.

Solid or liquid pharmaceutically acceptable carriers, diluents, vehicles, or excipients may be employed in the pharmaceutical compositions. Illustrative solid carriers include starch, lactose, calcium sulfate dihydrate, terra alba, sucrose, talc, gelatin, pectin, acacia, magnesium stearate, and stearic acid. Illustrative liquid carriers include syrup, peanut oil, olive oil, saline solution, and water. The carrier or diluent may include a suitable prolonged-release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., solution), or a nonaqueous or aqueous liquid suspension.

Administration of a PDGF receptor antagonist, especially compound I, and its pharmaceutically acceptable salts and solvates, may be performed according to any of the generally accepted modes of administration available to those skilled in the art. Illustrative examples of suitable modes of administration include oral, nasal, parenteral, topical, transdermal, and rectal.

A dose of the pharmaceutical composition contains at least a therapeutically effective amount of the active compound (e.g., compound I or a pharmaceutically acceptable salt or solvate thereof), and preferably is made up of one or more pharmaceutical dosage units. The selected dose may be administered to a mammal, preferably a human patient, in need of treatment by any known or suitable method of administering the dose, including: topically, for example, as an ointment or cream; orally, rectally, for example, as a suppository; parenterally by injection; or continuously by intravaginal, intranasal, intrabronchial, intraaural, or intraocular infusion.

A “therapeutically effective amount” is intended to mean the amount of an active agent that, when administered to a mammal in need thereof, is sufficient to effect treatment of cell proliferative diseases. The amount of a given compound that will be therapeutically effective will vary depending upon factors such as the particular compound, the disease condition and the severity thereof, the identity of the mammal in need thereof, which amount may be routinely determined by artisans.

“Treating” or “treatment” of a disease state includes:
(1) preventing the disorder, i.e., causing the clinical symptoms of the disease state not to develop in a mammal, preferably a human, subject that may be exposed to or predisposed to the disease state, but does not yet experience or display symptoms of the disease state;
(2) inhibiting the disease state, i.e., arresting the development of the disease state or its clinical symptoms; or
(3) relieving the disease state, i.e., causing temporary or permanent regression of the disease state or its clinical symptoms.
(4)

A <<disease state>> as used above refers to a cell proliferative disease implying accumulation of a given type of cells, and includes all tumors, cancers, carcinomas, sarcomas, lymphomas, blastomas, and the like. Preferably, the cell proliferative disease is a glioblastoma.

The terms “genetic marker”, “biomarker”, “marker”, and “feature” are synonymous and are used herein interchangeably.

Compound I is 4-(4-methylpiperazin-1-ylmethyl)-N-[4-methyl-3-(4-pyridin-3-yl)pyrimidin-2-ylamino)phenyl]-benzamide having the following formula

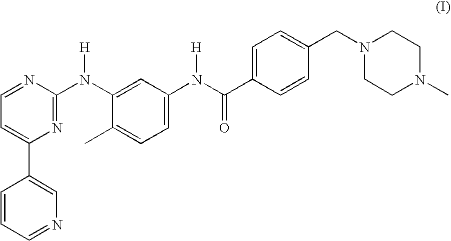

Compound I free base, pharmaceutically acceptable salts thereof, as well as preparation thereof, are disclosed in granted European patent EP 0564409, hereby incorporated by reference. Compound I free base corresponds to the active moiety. Compound I is an inhibitor of platelet-derived growth factor receptors alpha and beta (PDGFRs [symbol] and β), Bcr-Abl and c-kit tyrosine kinases.

The monomethanesulfonic acid addition salt of compound I, hereinafter referred to as “salt I”, and a preferred crystal form thereof, e.g., the beta crystal form, are described in granted European patent EP 0998473, hereby incorporated by reference.

A first aspect of the present invention thus concerns a method for in vitro diagnosing a cell proliferative disease in a mammal, comprising at least:

a) providing a biological sample from said mammal; and
b) determining the expression and/or phosphorylation profile in said sample of at least 2 to 40 genetic markers selected from Table 3.

Advantageously, the expression and/or phosphorylation profile of at least 3 to 5 genetic markers only, said markers being selected from Table 3, is determined in step b).

Levels of expression and/or phosphorylation state of genetic markers may be assayed in the biological sample by any conventional technique based on, e.g., RNA expression using for example the technique of RT-PCR, or based on, e.g., protein expression using for example any technique among Western blotting, immunohistochemistry or ELISA (enzyme-linked immunosorbent assay), including immunoassays, immunoprecipitation and electrophoresis assays. Preferably, the skilled artisan will determine the level of expression of genetic markers and/or the level of phosphorylation thereof in the sample.

For example, antibodies specific for genetic markers in their nonphosphorylated form, or in their phosphorylated form, or in both nonphosphorylated and phosphorylated forms, can be used in standard immunoassa to measure the expression and/or phosphorylation levels of said markers. ELISA-type assays, immunoprecipitation-type assays, conventional Western blotting assays, and immunohistochemistry assays using for example monoclonal or polyclonal antibodies can also be used for determining levels of expression and/or phosphorylation of markers.

According to a second aspect, the present invention relates to a method for predicting the behaviour of a mammal having a cell proliferative disease, in response to a medical treatment using at least one PDGF receptor antagonist, comprising at least:

a) providing a biological sample from said mammal;
b) determining the expression and/or phosphorylation profile in s aid sample of at least 2 to 40 genetic markers selected from Table 3;
c) comparing the expression and/or phosphorylation profile obtained in step b) to the means ±standard deviations calculated from Table 3 for responsive and non-responsive expression and/or phosphorylation profiles; and
d) predicting the behaviour of said mammal as follows:
when the expression and/or phosphorylation profile obtained in b) is in the mean ±standard deviation calculated for responsive expression and/or phosphorylation profiles, then said mammal is predicted to be responsive to said treatment;
when the expression and/or phosphorylation profile obtained in b) is in the mean ±standard deviation calculated for non-responsive expression and/or phosphorylation profiles, then said mammal is predicted to be non-responsive to said treatment; and
when the expression and/or phosphorylation profile obtained in b) is out of the means ±standard deviations calculated for responsive and non-responsive expression and/or phosphorylation profiles, then the behaviour of said mammal in response to said treatment is undetermined.

In a particular embodiment, the expression and/or phosphorylation profile of at least 3 to 5 genetic markers only, selected from Table 3, is determined in step b).

According to a third aspect, the present invention is directed to a method for selecting a mammal having a cell proliferative disease, wherein said mammal is predicted to be responsive to a medical treatment using at least one PDGF receptor antagonist, comprising at least:

a) predicting the behaviour of said mammal by using a method as described above; and
b) if said mammal is predicted to be responsive, then selecting said mammal.

This mammal may be selected for various purposes such as for entering a clinical trial or for being administered a medical treatment using at least one PDGF receptor antagonist or pharmaceutically acceptable salt thereof.

A fourth aspect of the present invention is related to a kit for in vitro analyzing the expression and/or phosphorylation profile of genetic markers in a mammal, said kit comprising cDNAs and/or antibodies for at least 2 to 40, preferably 3 to 5, genetic markers selected from Table 3.

In a fifth aspect, the present invention concerns a microarray or a biochip for in vitro analyzing the expression and/or phosphorylation profile of genetic markers in a mammal, comprising cDNAs and/or antibodies for at least 2 to 40, preferably 3 to 5, genetic markers selected from Table 3.

A sixth aspect of the invention relates to the use of at least one gene and/or at least one gene product selected from Table 3 as a genetic marker for:
in vitro diagnosing a cell proliferative disease in a mammal; and/or
predicting the behaviour of a mammal having a cell proliferative disease in response to a medical treatment using at least one PDGF receptor antagonist; and/or
selecting a mammal having a cell proliferative disease, wherein said mammal is predicted to be responsive to a medical treatment using at least one PDGF receptor antagonist.

In this respect, cDNA corresponding to said gene, and/or antibody(ies) specific for said gene product (in its phosphorylated form, or in its nonphosphorylated form, or both) is advantageously used.

According to a seventh aspect, the present invention is directed to the use of the aforementioned kit, microarray or biochip for:
in vitro diagnosing a cell proliferative disease in a mammal; and/or
predicting the behaviour of a mammal having a cell proliferative disease in response to a medical treatment using at least one PDGF receptor antagonist; and/or
selecting a mammal having a cell proliferative disease, wherein said mammal is predicted to be responsive to a medical treatment using at least one PDGF receptor antagonist.

According to an eighth aspect, the present invention is related to the use of at least one PDGF receptor antagonist for the manufacture of a drug for treating a responsive mammal having a cell proliferative disease, wherein said responsive mammal is selected using the method described above.

The invention also discloses a method for treating a cell proliferative disease in a responsive mammal in need of such treatment, comprising administering thereto a therapeutically effective amount of a PDGF receptor antagonist, said responsive mammal having being selected by using a method as previously described.

In a particular embodiment, said PDGF receptor antagonist is comprised in a pharmaceutical composition.

The present invention is illustrated, while not being limited, by the following figures:

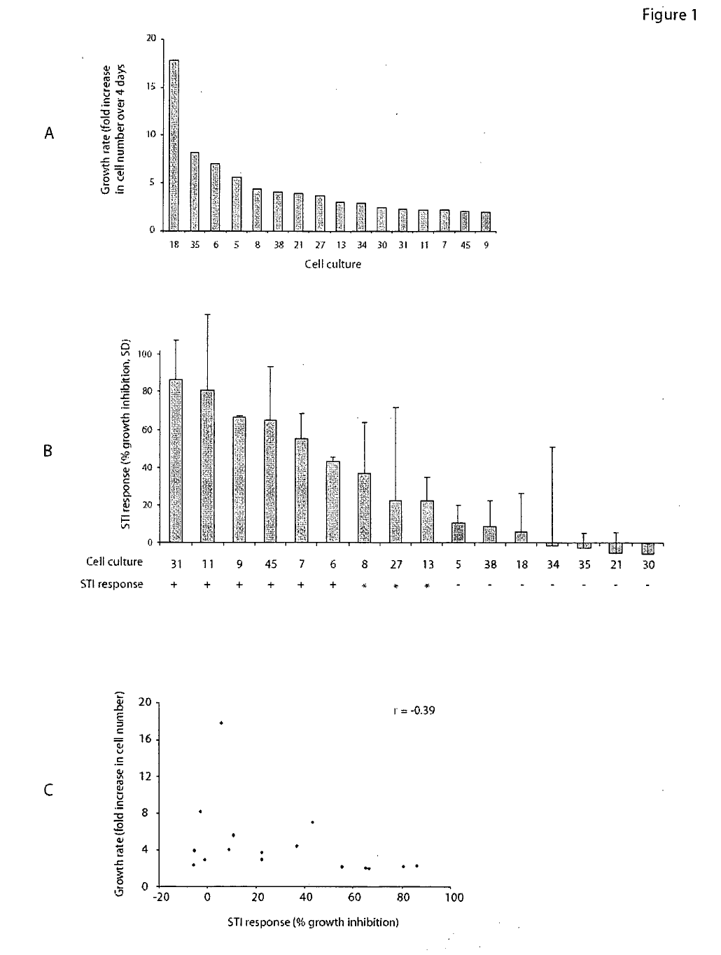

FIG. 1. Growth rate and compound I-senstivity of GBM cultures.

(A) Growth rates of 23 GBM cultures were determined by seeding 4000 cells/well in 24-well plates, and determining cell numbers after 4 days of culture. Values represent fold of growth over the culture period and represent the average value from two independent experiments. (B) For determination of sensitivity to compound 1,4000 cells of each of 16 GBM cultures, excluding the 7 most slow-growing cultures, were seeded in 96-well plate wells and grown for 4 days in the presence or absence of 1 μM compound I. Cell number at the end of the culture was determined by staining with crystal violet and photometric measurement. compound I-sensitivity is expressed as percent inhibition of growth induced by compound I treatment. Results, presented with standard deviation, are derived from 3 independent experiments in which each culture was analyzed in quadruplicates. (C) Correlations between growth-rate and compound I-sensitivity is illustrated by presenting results in a scatter plot showing a Pearson's correlation coefficient of 0.39.

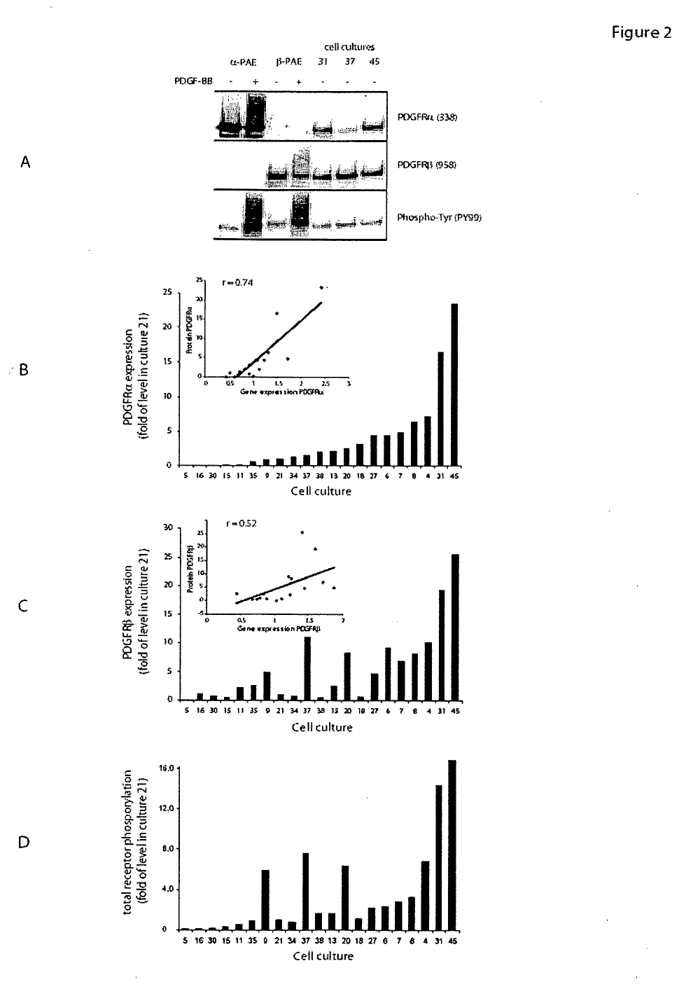

FIG. 2. PDGF receptor expression and activation in GBM cultures.

The expression levels and activation status of PDGF α- and β-receptors in the different cell cultures was determined by consecutive immunoblotting of WGA-fractions from GBM cultures with antibodies recognizing PDGFRα, PDGFRβ and phosphotyrosine. Samples from cells expressing either receptor were used as specificity controls and for normalization between different filters. (A) Representative example of immunoblotting analyses of GBM cultures and control cells. Expression of PDGF α-receptor (B) and PDGF β-receptor (C) in the GBM cultures. Cell lines are ordered according to levels of PDGF α-receptor expression. Expression level in GBM culture 21 was arbitrarily set to 1. Insets show the correlation between expression of the PDGF α- and β-receptors as determined by immunoblotting and mRNA expression analyses (Pearson's correlation, PDGF α-receptor r=0.86, PDGF β-receptor r=0.52). (D) Tyrosine phosphorylation of the PDGFRs as determined by phospho-tyrosine immunoblotting. Areas of the filters corresponding to the combined migratory positions of the PDGF α- and β-receptors were analyzed. The cell lines are ordered as in B and C. Total PDGF receptor phosphorylation in GBM culture 21 was arbitrarily set to 1.

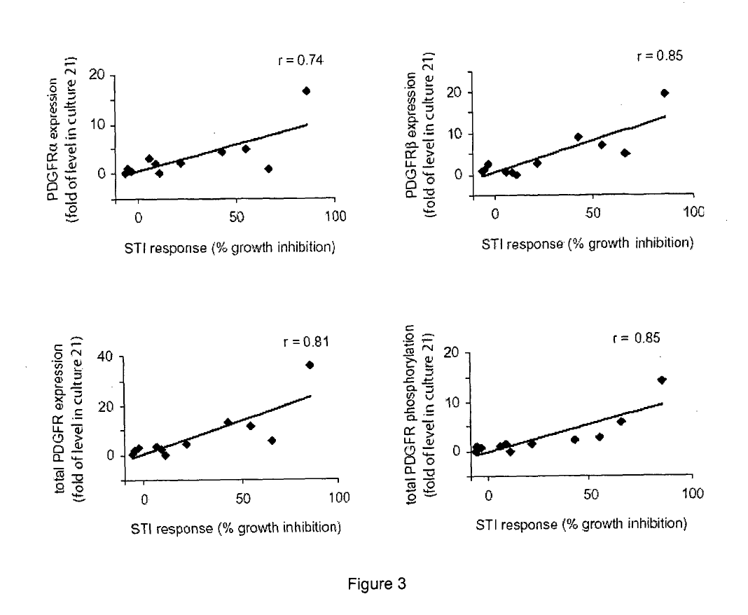

FIG. 3. Correlations between compound I-sensitivity and PDGFR status.

Correlations between compound I sensitivity and PDGF preceptor expression (upper left panel), PDGF β-receptor expression (upper right panel), combined PDGF α- and β-receptor expression (lower left panel) and total PDGF receptor tyrosine phosphorylation (lower right panel) are shown. Analyses were performed on the 11 GBM cultures remaining after exclusion of the 5 GBM cultures which showed the largest inter-experimental variation in the compound I-sensitivity experiments.

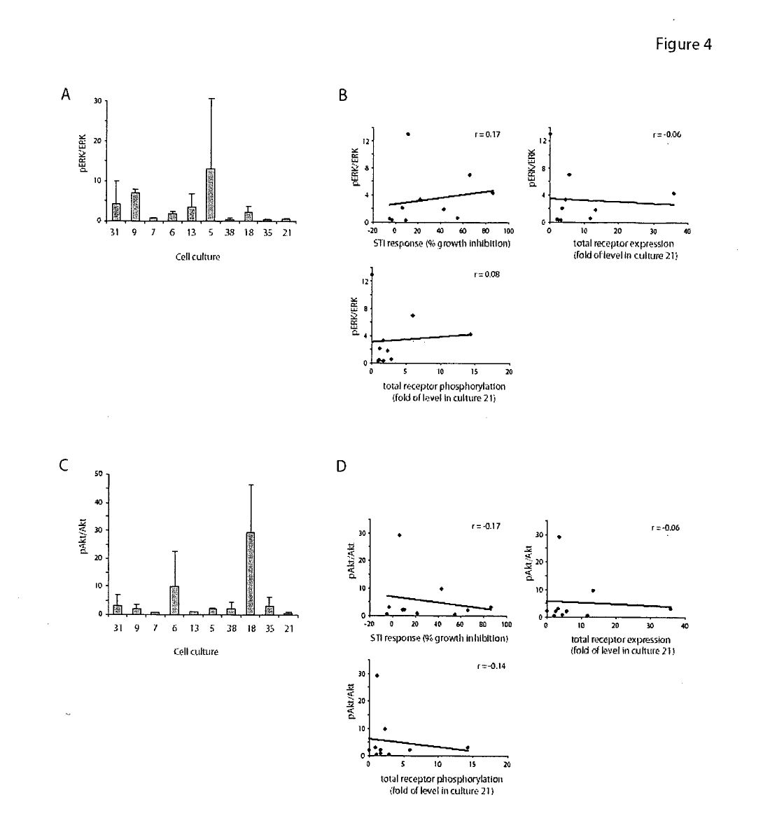

FIG. 4. Phosphorylation levels of ERK and Akt in GBM cultures and correlations between these parameters and compound I sensitivity or PDGF receptor status.

Specific phosphorylation of ERK and Akt were determined by immunoblotting using antibodies recognizing p44/42 MAPK, phospho-p44/42 MAPK Thr202/Tyr204, Akt and phospho-Akt Ser473. ECL signals were quantified and normalized for differences in transfer efficiency between filters by using the control lysates. Relative phosphorylation of ERK (A) and Akt (C) in 10 GBM cultures and correlations between the phosphorylation of ERK, Akt and compound I sensitivity (B,D, upper left panels), PDGF receptor expression (B,D, upper right panels) and PDGF receptor phosphorylation (B, D, lower panels).

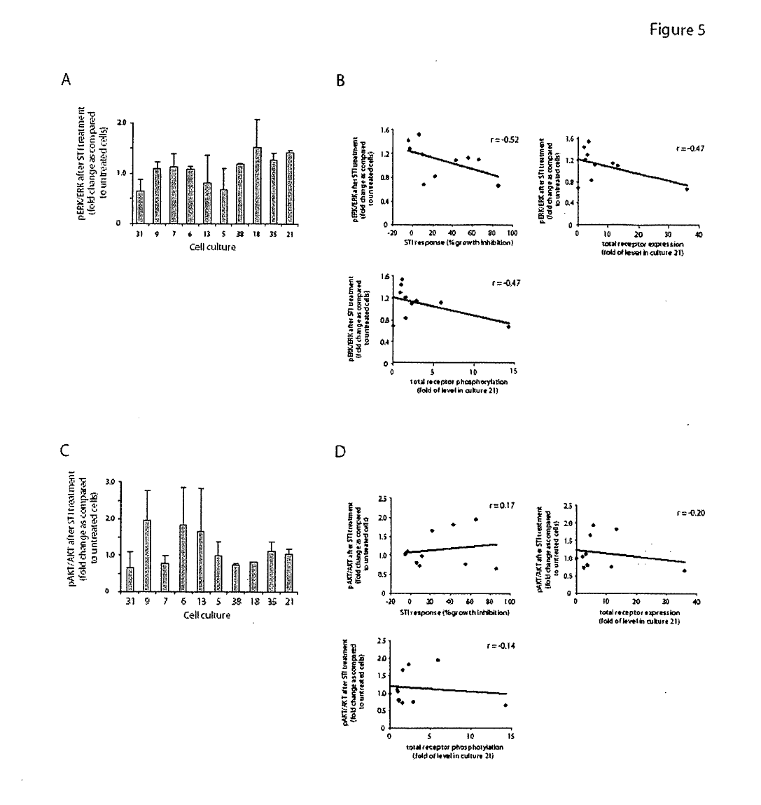

FIG. 5. Analyses of effects of compound I on phosphorylation of Akt and ERK, and correlation between these parameters and compound I sensitivity and PDGF receptor status.

Compound I-induced changes in ERK and Akt was monitored by comparing phosphorylation of ERK and Akt in un-treated cells and cells preincubated with 1 μM of compound I for 1 h. Compound I-induced changes in phosphorylation of ERK (A) and Akt (C) in 10 GBM cultures and correlations between these parameters and compound I sensitivity (B,D, upper left panels), PDGF receptor expression (B,D, upper right panels) and PDGF receptor phosphorylation (B, D, lower panels).

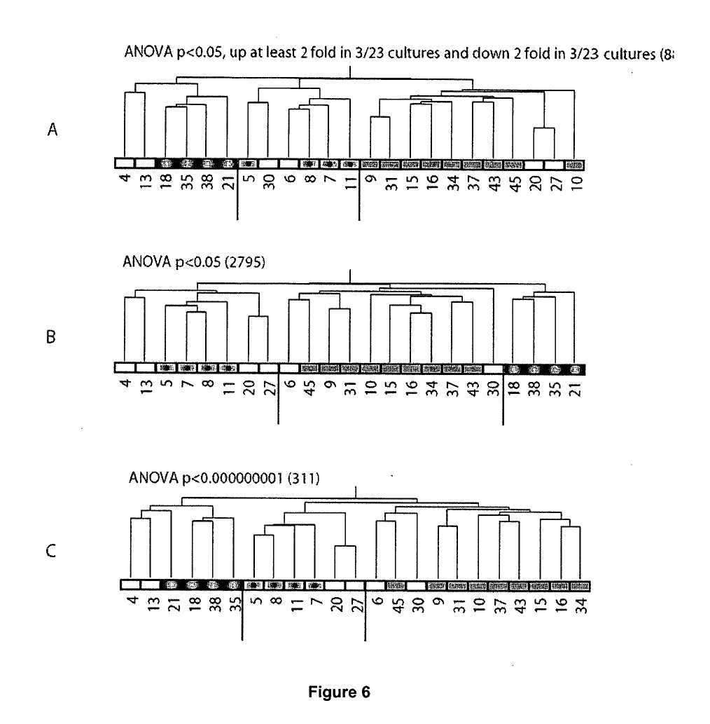

FIG. 6. Hierarchical clustering of 23 glioblastoma cell cultures with three different criteria for selection of features used in the clustering analysis.

(A) Hierarchical clustering by Pearson's correlation with a gene list containing 88 elements having a p-value less than 0.05 in an ANOVA test and also showing more than 2-fold up-regulation in at least 3 GBM cultures and 2-fold down-regulation in at least 3 other GBM cultures. (B) Clustering of GBM cultures with a 2795 feature list, obtained by setting a significance level of 0.05 in an ANOVA test (C) Clustering after generation of list of 311 features, obtained as in B, but with a setting of significance according to ANOVA test to p<0.000000001. Color coding is used to illustrate that regardless which criteria that was used for selection of feature list, three major clusters were formed which in all cases showed the same distribution of 17 out of the 23 GBM cultures, e.g. GBM cultures 5, 7, 8 and 11 always clustered together.

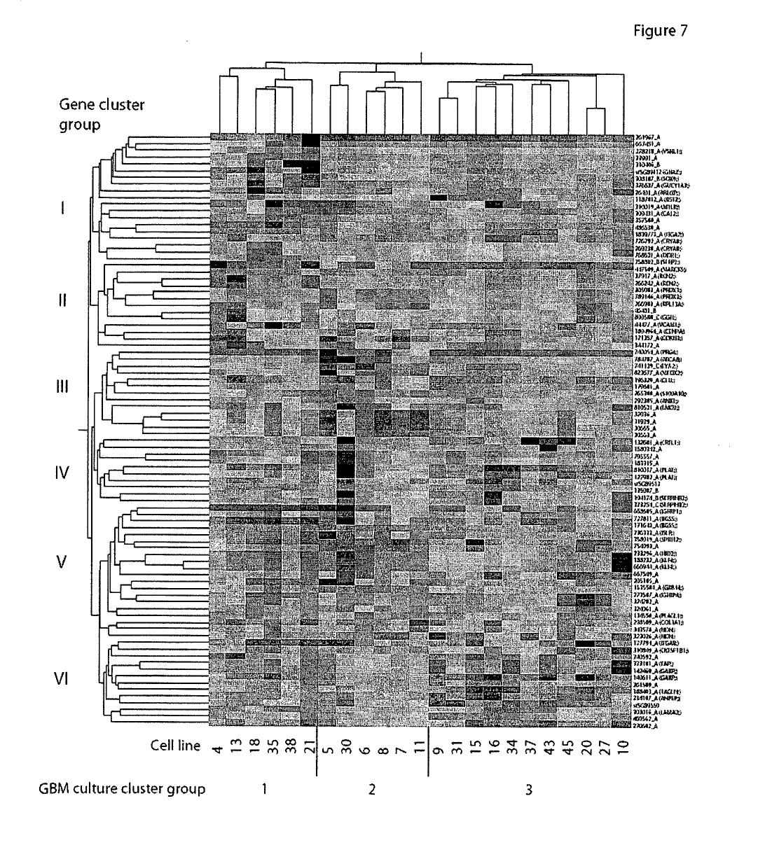

FIG. 7. Clustering of the genes that define the three subgroups of GBM cultures.

The features used for the GBM cell cluster illustrated in FIG. 6A were hierarchically clustered by Pearson's correlation giving a relationship tree for the features. This clustering analyses groups genes according to similarities in expression pattern across the 23 GBM cultures. Red and green color indicates high and low expression, respectively, of the genes in the individual GBM cultures.

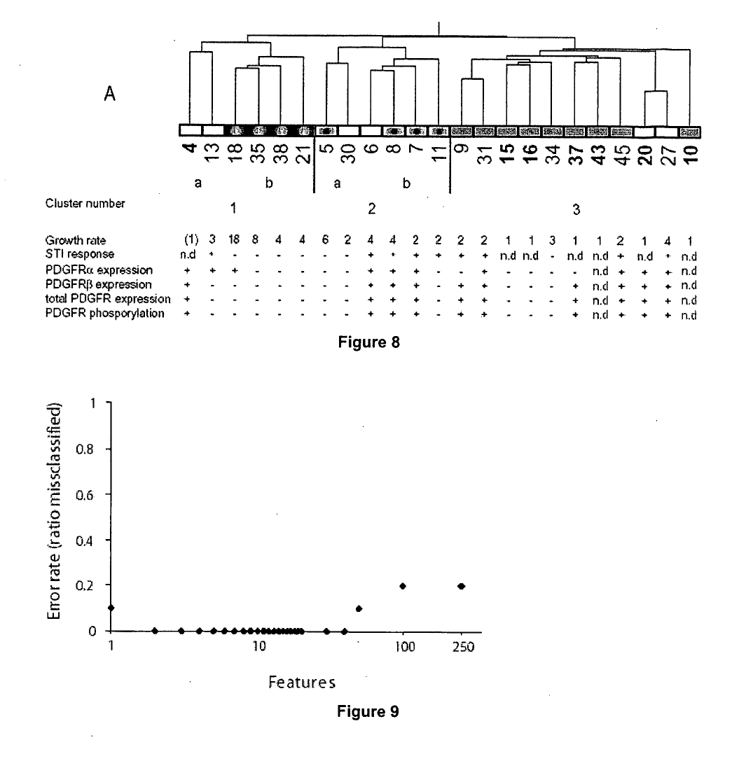

FIG. 8. Compilation of results obtained after biochemical characterization, and expression profiling, of the 23 GBM cultures.

The clustering shown is the one obtained after selection of features which show a p-value less than 0.05 in an ANOVA test and also showing more than 2-fold up-regulation in at least 3 GBM cultures and 2-fold down-regulation in at least 3 other GBM cultures (FIG. 6A). Description of growth rate is as in FIG. 1A with numbers indicating fold increase in cell number over a four-day culture period. Concerning compound I sensitivity the 16 analyzed GBM cultures were divided into 6 responders (+, showing more than 40% growth inhibition) 7 non-responders (−; showing less than 20% growth inhibition) and three intermediate responders (*; 20-40% growth inhibition). For PDGF receptor expression and phosphorylation, the 21 analyzed GBM cultures were divided into two groups with high (+; 10 GBM cultures) or low (−; 11 GBM cultures) PDGF receptor expression or phosphorylation.

FIG. 9. Performance of a weighted-voting classification of compound I responders and non-responders in a leave-one-out test.

Cell lines 6, 7, 9 and 31 were chosen as responders and 5, 18, 21, 30, 35 and 38 as non-responders for the classification. x-axis describes number of features used for classification (1-250), and y-axis describes the fraction of misclassified cultures in the leave-one-out tests.

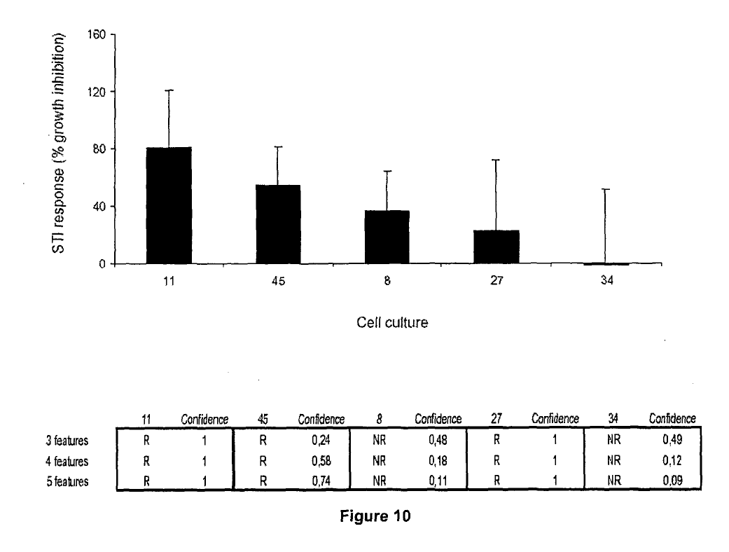

FIG. 10. Performance of classifiers on five GBM cultures excluded from the training set.

Classifiers, composed of 3-5 features, generated from the top features in a signal to noise ranked gene list from 10 glioblastoma cell, were used to predict response of 5 additional GBM cultures. Staple diagram shows the compound I sensitivity of the cultures as determined in FIG. 1B. Below each bar is given the classification of the 5 GBM cultures, obtained with feature lists composed of 3, 4 or 5 features. Strength of prediction with the different classifiers, for each cell culture, is given by the confidence value.

The present invention will be better understood in the light of the following detailed description of experiments, including examples. Nevertheless, the skilled artisan will appreciate that this experimental description is not limitative and that various modifications, substitutions, omissions, and changes may be made without departing from the scope of the invention.

### EXAMPLES

### I—Materials and Methods

### I-1—Tissue Culture and Determination of Growth Rate and Compound I-Sensitivity of GBM Cultures

Establishment of primary GBM cultures was performed according to standard procedures (Ponten and Westermark, 1978). Primary cell cultures derived from glioblastomas were grown in an atmosphere of 5% CO₂ at 37° in MEM supplemented with 10% FBS, 10 U/ml penicillin and 10 μg/ml streptomycin.

For determination of growth rate, cells were seeded at a density of 4000 cells per well in 24 well plates (Sarstedt). After 4 days of culture, cells were harvested by trypsin digestion and counted in a Coulter cell counter. Growth rate was expressed as fold-increase in cell number during the culture period. The data presented are derived from two independent experiments, in which each analysis was performed in duplicates.

### I-2—Growth Inhibition Induced by Compound I

Compound I was obtained from Novartis Pharmaceuticals. For each experiment fresh 1 mM compound I stock solutions were prepared by dissolving 6 mg compound I in 10 ml PBS followed by sterile filtration with a 45 μm filter. Cell cultures having a growth rate not exceeding 1.2 fold over a four day period were not tested for growth inhibition induced by compound I. For determination of the effect of compound I on cell growth, cells were seeded at a density of 4000 cells per well in 96 well plates (Sarstedt). The following day media was exchanged to media with or without 1 μM compound I. After 4 days of incubation, including media change after two days, cells were fixed for 30 min in cold 4% paraformaldehyde (PFA) in PBS, and stained 30 min with 0.01% crystal violet in 4% ethanol. Samples were washed 3 times with tap water, and air dried for at least 30 min. Stained cells were dissolved in 100 μl 1% SDS and absorbance was quantified with a Biomek 1000 (Beckman) optical tool using the 600 nm filter.

Effects of compound I were expressed as % reduction of increase in cell number during the four-day period of treatment, thus 100% growth reduction corresponds to a situation with equal number of cells at end and start of the culture period. As positive control, each experiment included cells which had previously been shown to display compound I-sensitive growth (Sjöblom et al., 2001). Data presented are derived from two or three independent analyses, performed with duplicates/quadruplicates, of the effects of compound I on each GBM culture.

### I-3—Preparation of Control Cell Lysates for Analyses of Expression and Phosphorylation Status of PDGF Receptors, ERK and Akt

Porcine aortic endothelial cells, stable transfected with the PDGF α- or β-receptor (PAE/R α and PAE/R β cells, respectively (Claesson-Welsh et al., 1988; Claesson-Welsh et al., 1989)) were seeded at high density in 10 cm dishes (Sarstedt) using standard culture conditions. After 16 h, cells were serum-starved, by exchange to medium containing 0.1% FBS, for 24 h. Cells were then treated for 5 min at 37° with or without 100 ng/ml PDGF-BB in medium containing 0.1% FBS. After washing with ice cold PBS, cells were lysed on ice for 10 min in 1 ml lysis buffer composed of 0.5% Triton X-100, 0.5% deoxycholic acid, 150 mM NaCl, 20 mM Tris pH 7.5, 10 mM EDTA, 30 mM tetra-sodium diphosphate decahydrate, 1% Trasylol, 0.5% phenylmethyl sulphonyl fluoride (PMSF) and 0.5% NaVO₃. After centrifugation for 15 min at 15000×g, cell lysates were collected and protein concentration was measured with the BCA protein assay reagent A kit (Pierce). After normalization of protein concentration, glycoproteins were isolated by incubation with wheat germ agglutinin (WGA)-sepharose for 16 h at 40. Samples were centrifuged for 15 min at 15000×g to pellet the W GA-sepharose beads. Supernatants were removed and saved as controls for ERK and Akt analysis. The WGA beads were washed 3 times with 1 ml high salt lysis buffer composed of 0.5% Triton X-100, 0.5% deoxycholic acid, 500 mM NaCl, 20 mM Tris pH 7.5, 10 mM EDTA, 30 mM tetra-sodium diphosphate decahydrate, 1% Trasylol, 0.5% PMSF and 0.5% NaVO₃. Cell lysate-supernatant or WGA-Sepharose fractions of glycoproteins were mixed with Laemmli buffer (0.0625 M Tris-HCl, 10% glycerol, 2% SDS, 5% beta-mercaptoethanol, 0.0125% bromophenol blue), heated to 95° for 5 min, and stored at −20°.

### I-4—Analysis of PDGF Receptor Expression and Phosphorylation

Cell lysates from approximately 500,000 GBM cells, derived from subconfluent cultures kept in medium supplemented with 10% FCS, were prepared as described above. WGA-fractions from cell lysates, with normalized protein content, were isolated as described above.

Samples were subjected to SDS-PAGE using 7% polyacrylamide gels. On each gel, control samples from un-stimulated or PDGF-BB-stimulated PAE/R α and PAE/R β cells were loaded. Proteins were subsequently electrophoretically transferred to Hydrobond-C-Extra filters (Amersham Life Science). For detection of PDGFRβ, the filters were blocked for 1 h in TBS containing 5% BSA, followed by over night incubation with primary PDGFR β antibody solution with 1 μg/ml 958 (Santa Cruz Biotechnologies) in TTBS (10 mM Tris-HCl, pH 7.4, 150 mM NaCl, 0.02% Tween-20). After three 10 min washes, the filters were incubated for 1 h with horseradish peroxidase coupled donkey anti-rabbit antibody (Amersham Life Science) diluted 1:25000 and washed three times in TTBS. The antigens were detected by enhanced chemoluminescence using the Lumi-Light plus Western blotting substrate (Roche) according to the manufacturer's instruction with an Intelligent Darkbox II digital scanner (FUJIFILM). Following detection the filters were stripped for 30 min at 50° in stripping buffer (2% SDS, 62.5 mM Tris HCl pH 6.7 and 100 mM beta-mercaptoethanol), washed once in TTBS and blocked for 1 h in TBS containing 5% BSA. For detection of PDGFRα the filters were re-probed with 1 μg/ml PDGFR α antibody 338 (Santa Cruz Biotechnologies) in TTBS and incubated overnight. Development and detection was performed as describe above. For detection of phosphorylated PDGFR the filters were re-probed a third time with 1 μg/ml phosphotyrosine specific antibody PY99 (Santa Cruz Biotechnologies) in TTBS and incubated overnight. Development and detection was performed as described above, with the exception of using horseradish peroxidase coupled sheep anti-mouse antibody (Amersham Life Science), diluted 1:50000 in TTBS, as secondary antibody.

Receptor expression and phosphorylation were quantified using the AIDA software version 3.10.039 (FUJIFILM). Differences between filters, in transfer efficiency were normalized for by relating values from the GBM cultures with those from the control samples. The PDGFR α and PDGFR β expression levels, and the receptor phosphorylation, in GBM culture 21 were arbitrarily given the value 1.

### I-5—Analysis of Akt and ERK Expression and Phosphorylation in GBM Cultures Grown in the Absence or Presence of Compound I

Glioblastoma cell cultures were confluently plated in 12-well plate wells (Falcon). The following day cells were left untreated or treated for 1 h with 1 μM compound I. Cell lysates were prepared as described above.

Samples, with normalized protein content, were subjected to SDS-PAGE using 12% gels. Control lysates from un-stimulated and PDGF-BB-stimulated PAE/Rβ cells were loaded on each gel. Samples were electrophoretically transferred to Hydrobond-C-Extra filters (Amersham Life Science). For detection of phosphorylated forms of ERK and Akt, filters were blocked for 1 h in Tris buffered saline pH 7.6 (TBS), 0.137 M NaCl and 0.0035 M Tris-HCl, containing 5% BSA, and incubated over night with 1 μg/ml anti phospho-p44/42 MAPK Thr202/Tyr204 (Cell Signaling Technology) or 1 μg/ml anti phospho-Akt Ser473 (Cell Signaling Technology) in TBS with 0.001% Tween-20 (TTBS). After three 10 min washes in TTBS, the filters were incubated for 1 h with horseradish peroxidase coupled sheep anti-rabbit antibody (Amersham Life Science) diluted 1:25000, and washed three times 10 min in TTBS. The antigens were detected using the Lumi-Light plus western blotting substrate (Roche) according to the manufacturer's instruction with an Intelligent Darkbox II digital scanner (FUJIFILM). After detection the filters were stripped for 10 min in 0.4 M NaOH, washed once in TTBS and blocked for 1 h in TBS containing 5% BSA. For determination of ERK and Akt expression, the filters were re-probed with 1 μg/ml anti p44/42 MAPK (Cell Signaling Technology) and 1 μg/ml anti Akt (Cell Signaling Technology) in TTBS overnight. Development and detection was performed as mentioned above. Values were quantified by using the AIDA software version 3.10.039 (FUJIFILM).

The relative expression and phosphorylation of ERK and Akt in the different GBM cultures, was determined by using the reference samples from the PAE/Rβ cells. Response to compound I treatment was expressed as fold change in specific phosphorylation of ERK and Akt.

### I-6—RNA Extraction for Analysis of Gene Expression

RNA was extracted, using the RNAeasy kit (Qiagen) according to the manufacturer's instructions, from one subconfluent 75 cm² culture dish of each of the 23 GBM cultures. RNA amounts were assessed spectrophotometrically, revealing yields of 10-100 μg RNA from the different cultures. Structural integrity of RNA was confirmed by agarose gel electrophoresis.

### I-7—Amplification and Labelling of RNA for Competitive Hybridization

5 μg of RNA from each cell line was used for linear amplification (Van Gelder et al., 1990) with some modifications. Shortly, cDNA was reversely transcribed in a mixture of 5 μg RNA, 1 μl bacterial RNA cocktail, 1 μl dT-T7 primer (1 μg/ml, SEQ ID No 1: AAA CGA CGG CCA GTG AAT TGT AAT ACG ACT CAC TAT AGG CGC TTT TTT TTT TTT TTT), 4 μl 5× Superscript II reaction buffer (Invitrogen), 2 μl DTT (Invitrogen), 1 μl Ultrapure dNTP mix (Clontech), 1 μl RNAsin (Ambion), 1 μl template switch oligo primer (1 μg/ml, SEQ ID No 2: AAA CAG TGG TAT CAA CGC AGA GTA CGC GGG) and 2 μl Superscript II (Invitrogen) at 420 for 1 h. For second strand synthesis, 106 μl water, 15 μl Advantage 10×PCR buffer (Clontech), 3 μl of Ultrapure dNTP mix, 1 μl RNase H (Promega) and 3 μl cDNA polymerase (Clontech) was added. Samples were then incubated in PCR machine (Applied Bio9systems) at 37° for 2 min for RNase H degradation. Samples were subsequently incubated at 94° for 3 min for denaturation, at 650 for 3 min for primer annealing and 75° for elongation by the cDNA polymerase. Reaction was stopped by addition of 7.5 μl of 1 M NaOH and 2 mM EDTA and incubated at 65° for 10 min. The reaction mixture was cleaned by phenol extraction with 350 μl water and 500 μl phenol-chloroform-isoamyl alcohol 25:24:1 (Sigma), washed three times in a Microcon YM-100 centrifugal filter with 500 μl water, and finally concentrated to a volume of 16 μl. Anti-sense RNA was generated by in vitro transcription with an in vitro transcription kit (Ambion). From the anti-sense RNA, cDNA was again reversely transcribed in a Superscript II reaction, as described above, followed by generation of double stranded DNA in a DNA polymerase reaction. In the second step of amplification UTP nucleotide mix was partly substituted with a 1:3 mix of UTP Cy-dye-UTP (Ambion). In parallel, a pool, composed of equal amounts of RNA from all the cultures, was amplified to be used as reference in the following hybridization experiments.

I-8—Hybridization of Cy-Dye-Labelled Anti-Sense RNA to Sense cDNA Chips

Each cell culture was hybridized, together with the reference sample of the pooled cultures, in quadruplicate as duplicate dye-swaps. For each hybridization, 4 μg labelled sample and 4 μg labelled pool in a volume of 66 μl was mixed with 4 μl cotDNA (1 mg/ml, Invitrogen), 4 μl poly adenylic acid (2 μg/ml, Sigma), 8 μl 70% ethanol and 7 μl 3 M sodium acetate pH 5.2. After precipitation, by incubation for 30 min. at −70°, samples were centrifuged at 15000×g for 20 min at 4°. Pellets were washed with 70% ethanol and air dried for 60 min, dissolved in 8 μl water and 40 μl hybridization solution (5×SSC, 6×Denhardt's solution, 60 mM Tris-HCl pH 7.6, 0.12% sarkosyl, 48% formamide, sterile filtered), heated 1000 for 5 min and cooled down to room temperature. Samples were placed on a pre-cooled microarray chip (The Welcome Trust Sanger Institute, Human version 1.2.1, containing roughly 10 000 elements, corresponding to about 6000 individual genes, http://www.sanger.ac.uk/Projects/Microarrays/) and covered by a cover glass and incubated for 16 h at 47° in a Corning hybridization chamber with 40 μl 40% formamide and 2×SSC at 47°. The chips were washed once in 2×SSC for 5 min, three times in 0.1×SSC and 0.1% SDS for 30 min and once in 0.1×SSC for 10 min. The chips were finally dried by centrifugation at 1000 rpm for 2 min.

### I-9—Array Scanning of the Chips and Data Extraction

Chips were scanned with a ScanArray 5000 (GSI Lumonics) using the ScannArray software version 3.1 (Packard BioChip Technologies). Expression intensity values were quantified by using the QuantArray software version 3.0.0.0 (Packard BioChip Technologies). Unreliable spots were flagged manually and signals were quantified by the histogram method.

### I-10—Hierarchical Clustering

Data was loaded into GeneSpring and then LOWESS normalized. Lists containing differentially expressed genes were generated by analysis of variance, ANOVA, or by arbitrarily set cut-off values. For analysis of variance in GeneSping, the global error model was turned off, since samples were not assumed to have the same variance, and the Bonferroni model was used for multiple testing correction. Feature lists were generated in several different ways but 3 versions were chosen for final presentation. In the first gene list, features had p-values less than 0.05 in an ANOVA test, and also had to fulfil the criteria of being up-regulated more than 2-fold in at least 3 samples and down-regulated more than 2-fold in at least 3 samples, which gave 88 features. The second and third gene lists contains 2795 and 311 features and were generated with the inclusion criteria of ANOVA p-values less than 0.05 or 0.000000001, respectively. The three gene lists were subsequently used to hierarchically cluster the cell cultures according to Pearson's correlation.

### I-11—Supervised Analyses to Identify Marker Genes for Compound I-Responsiveness

The weighted voting method (Golub et al., 1999) was applied to the 10 cell cultures having the least inter-experimental variation in the growth inhibition experiments values. Expression data from the 10 cell cultures was loaded into GeneCluster version 2.1.3 beta (http://www-genome.wi.mit.edu/cancer/software/genecluster2/gc2.html) (Golub et al., 1999; Tamayo et al., 1999). Classification performance with feature lists of different length were tested by leave-one-out cross validation. The choice of classifying features is based on using the allowed number of features having the highest median signal to noise values. GeneCluster was set to pick features with the highest absolute signal to noise value, not requiring the lists to contain the same number of features from the positive and negative side of the signal to noise ranked gene list.

For evaluation of classifiers on an independent set of GBM cultures, classifiers with 3-5 features were built. Feature selection was based on highest signal to noise ratio of features among the responders and non-responders in the training set. These classifiers were then used for a classification, based on a weighted-voting procedure, of 5 independent GBM cultures for which compound I sensitivity had been determined empirically.

### II—Results

### II-1—Characterization of Compound I-Sensitivity of the GBM Cultures

Before characterizing the 23 different cultures with regard to compound I sensitivity, the growth properties of the individual cultures were analyzed by determining increase in cell number over a four-day period of growth in medium supplemented with 10% FCS. As indicated in FIG. 1A, big variations in growth rate were observed. The most slow-growing cultures only showed a 1.2-fold increase in cell number over the four day period, whereas the fastest growing culture displayed an 18-fold increase in cell number.

Growth inhibition induced by compound I treatment was analyzed by comparing cell number after four days of growth in the absence or presence of compound I. Effects of compound I were expressed as % reduction of increase in cell number during the four-day period of treatment. The 7 most slow-growing cultures were excluded from this analysis. Results from three independent experiments of the remaining 16 cultures are shown in FIG. 1B. Large differences in response to compound I treatment was observed between the cultures. The cell cultures 5, 18, 21, 30, 34, 35 and 38 all displayed less than 15% growth inhibition. In contrast the growth of cultures 6, 7, 9, 11, 31 and 45 was reduced more than 40%. Cultures 8, 13 and 27 exhibited an intermediate response of 20-40% growth inhibition.

To analyze if growth inhibition was related to growth rate, the correlation between these two parameters were calculated. As shown in FIG. 1C, this analysis did not provide any evidence for strong correlations between growth rate and response to compound I treatment.

II-2—PDGF Receptor Expression and Activation in the GBM Cultures and Correlation with Compound I Sensitivity

PDGF receptors are the most likely targets mediating the growth inhibitory action of compound I-induced growth inhibition of the GBM cultures. PDGF receptor expression and activation was therefore analyzed and these parameters were correlated with growth inhibition (FIGS. 2 and 3).

PDGF receptor activation and expression was analyzed by immunoblotting of WGA-fractions from cultured GBM cells with antibodies against PDGF α- and β-receptor and with phospho-tyrosine antibodies. As positive controls, ligand-stimulated or unstimulated porcine aortic endothelial cells transfected with PDGF α- or β-receptors were used (FIG. 2A). The value for receptor expression, and total PDGF receptor phosphorylation, was arbitrarily set to 1 in culture 21.

As shown in FIGS. 2B and C, more than 100-fold variation was observed in PDGF α- and β-receptor expression between the cultures. The estimates of PDGF receptor protein expression was compared with data from the gene expression analyses (see below) and resulted in r-values of 0.86 and 0.52 for the PDGF α- and β-receptors, respectively (FIGS. 2B and C, insets). Furthermore, PDGF receptor phosphorylation was determined by quantifying the phospho-tyrosine signal at the combined migratory positions of PDGF α- and β-receptors (FIG. 2A, D). Overall, this analysis yielded a pattern very similar to that obtained combining PDGF α- and β-receptor expression. Thus, this analysis indicated that cultures displayed similar phosphorylation per receptor.

The results from correlations of these data with the compound I sensitivity of the 11 of the 16 analyzed cultures are shown in FIG. 3. Cultures 11, 45, 8, 27 and 34 were omitted from this analysis due to the large variations in the growth inhibition experiments of these cultures. All four analyzed PDGF receptor-related parameters showed high correlations with compound I sensitivity, with r-values ranging between 0.85 (PDGF β-receptor expression) and 0.73 (PDGF α-receptor expression).

Thus, these analyses revealed a broad variation with regard to PDGF receptor expression within the panel of GBM cultures, and also revealed a strong correlation between PDGF receptor expression and compound I sensitivity and between total PDGF receptor phosphorylation and compound I sensitivity.

### II-3—Activation Status of ERK and Akt in the Absence and Presence of Compound I

The protein kinases ERK and Akt are important mediators of PDGF receptor signaling, but also participate in downstream signaling triggered by other types of cell surface receptors, e.g. integrins. Both enzymes are activated through phosphorylation, and immunoblotting with antibodies specific for the activated phosphorylated forms was therefore used for determination of activation status of these enzymes. The activation status of these enzymes was determined, in the 11 cultures with robust results from the growth inhibition studies. Activation status of both ERK and Akt showed big variations between cell cultures (FIGS. 4A and C). When activation status was correlated with compound I response (FIGS. 4B and D upper left panels), total PDGF receptor expression (FIGS. 4B and D, upper right panels) or PDGF receptor phosphorylation (FIGS. 4B and D, lower panels) no correlations were observed.

The changes in specific phosphorylation of ERK and Akt induced by one hour of treatment with compound I were analyzed to investigate if drug-induced alterations in these pathways correlated with compound I response or receptor expression. Overall, only moderate changes in ERK and Akt phosphorylation were observed after drug treatment (FIGS. 5A and C). No correlations were observed between compound I-induced alterations in Akt phosphorylation and compound I responsiveness or PDGF receptor status (FIG. 5D). However, a correlation between compound I-induced growth inhibition and reduction in ERK phosphorylation was observed (r=−0.47).

These analyses thus show that big variations in ERK and Akt activation occurred between the cell cultures and indicate that that basal levels of activation of these pathways are not correlated with PDGF receptor status or compound I sensitivity. They also establish that compound I treatment is not associated with strong alterations of the net activation status of these signaling molecules. However, some correlations between growth response and changes in compound I-induced changes in ERK phosphorylation were noted.

### II-4—Gene Expression-Based Clustering of 21 GBM-Derived Primary Cultures

To describe the gene expression-based differences and similarities between the 23 GBM-derived primary cultures, gene expression profiling was performed. RNA was isolated from low-passage cultures grown in 10% FCS. To obtain sufficient amounts of RNA for microarray analyses, RNA was subjected to two rounds of amplification. Fluorescent dyes were incorporated into the RNA during the second amplification. Each culture was analyzed in quadruplicates using a cDNA array containing approximately 10000 human cDNAs (http://www.sanger.ac.uk/Projects/Microarrays/). The reference RNA was composed of a pool of RNA from all of the cultures.

The results from the hierarchical clustering are shown in FIG. 6. As indicated, different statistical criteria were used for determining which genes that should be used for the clustering. Regardless of which criteria that were used some consistent patterns could be observed which involved 17 of the 23 samples. Cultures 18, 21, 35, and 38 clustered together in all analyses (cluster 1). Also, cultures 5, 7, 8 and 11 occurred together in all analyses (cluster 2). Finally, a group containing cultures 9, 10, 15, 16, 31, 34, 37, 43 and 45 (cluster 3) was seen regardless of statistical criteria for selection of genes.

FIG. 7 shows the clustering diagram, derived after analyses with genes showing at least two-fold regulation in three of the samples, with inclusion of the cluster-defining genes which are also listed in Tables 1 and 2.

In Table 1 the differentially expressed genes are grouped according to their molecular function as described by a gene ontology program. The 88 hybridization signals used for the clustering of cells represents 75 unique genes. Among these genes, 47 were ascribed a function in the gene ontology program: most of the genes were found in the categories signal transduction proteins, regulators of transcription, and proteins associated with adhesion or proliferation.

In Table 2 the genes are organized according to how they appear in the gene cluster that defines the three clusters of cultures. Their average expression in the three clusters of cultures is highlighted to illustrate their expression pattern in relation to the three clusters of cultures. In general, gene cluster groups III and VI are composed of genes that are up-regulated in culture clusters 2 and 3, respectively. Gene cluster group I is almost always high in culture cluster group 1 with some exceptions. Conversely, gene clusters IV and V contains genes that are down regulated in culture cluster 1, and gene cluster II is composed of genes with low expression in culture cluster 3.

II-5—Comparison of the Growth Properties and the PDGF Receptor Status with the Gene Expression-Based Clustering of the GBM Cultures

The results from the analysis of the growth rate of the GBM cultures, and their compound I sensitivity, were combined with the results from the gene expression-based hierarchical clustering of the cultures (FIG. 8). Some interesting tendencies were observed. Six of the seven most slow-growing cultures (marked in bold) occurred in cluster 3. The six responders were concentrated to clusters 2 and 3. Within cluster 2 they all occurred within sub-cluster 2b. Of the 7 non-responders, 4 cultures (18, 21, 35, 38) made up the complete set of sub-cluster 1b.

Also, the results from the PDGF receptor status were compared with the gene expression-based grouping of the cultures (FIG. 8). Cultures were categorized into two classes with regard to PDGF receptor expression and phosphorylation yielding 10 cultures displaying high expression or phosphorylation and 11 cultures with low expression or phosphorylation. Cluster 1 were clearly enriched for cultures with low receptor expression and phosphorylation, whereas both cluster 2 and 3 were composed of mixtures of the two categories. This compilation of the data also highlights that all six robust compound I non-responders showed low receptor expression and phosphorylation and, conversely, that all clear responders were characterized by high receptor expression and phosphorylation.

II-6—Supervised Identification of a Gene Expression Pattern Associated with compound I Responsiveness

In view of the indications that correlations existed between gene expression pattern and compound I responsiveness, a supervised analysis was performed with the aim of identifying the gene expression patterns that best correlated with compound I responsiveness. For that purpose the 10 cultures yielding the most clear results from the analyses of compound I sensitivity were divided into responders (cultures 6, 7, 9 and 31) and non-responders (cultures 5, 18, 21, 30, 35 and 38) (FIG. 1B).

Supervised analysis was performed using the method of weighted voting, including a “leave-one-out” validation. As shown in FIG. 9, when 2-40 features were used for classification, all 10 cultures were correctly classified in the leave-one-out validation. In total, 8 and 16 genes were used for the classification with lists composed of 4 and 10 features, respectively. The genes used in the leave-one-out test are tabulated in Table 3.

Using expression data from all 10 cultures, classifiers of 3-5 features were made (Table 4). Using this classifier, all 10 cultures used to build the classifier, were correctly described as responders and non-responders. To extend this analysis, the classifiers were used for a preliminary test on the five cultures (cultures 8, 11, 27, 34 and 45) not included in the training set (FIG. 10). The results from application of these classifiers on this preliminary test set are shown in FIG. 10. Interestingly cultures 11, 45 were with all three classifiers characterized as responders, in agreement with the results from the growth inhibition experiments. Also, in agreement with the growth inhibition results, culture 34 was consistently classified as non-responders. Cultures 8 and 27, showing intermediate responses, were with all three classifiers designated as non-responder and responder, respectively.

As shown above, primary GBM cultures vary extensively with regard to compound I-sensitivity. By characterization of PDGF receptor status, clear correlation between compound I-sensitivity and PDGF receptor expression and phosphorylation was shown (FIGS. 1-3, 8). No obvious correlations were seen between compound I-sensitivity or basal phosphorylation of ERK and Akt (FIG. 4). compound I-sensitivity showed some correlation with compound I-induced reduction in ERK phosphorylation. (FIG. 5). Gene expression profiling indicated the presence of distinct GBM subsets, which differed with regard to compound I-sensitivity, growth rate and PDGF receptor phosphorylation (FIGS. 6-8). Finally, using supervised analyses of gene expression data, short gene lists predicting compound I-response could be generated (FIGS. 9, 10).

Although previous studies have reported inhibition of GBM cell lines, a systematic analysis of the effects of PDGF receptor inhibition on GBM growth is describes herein for the first time.

The correlation between PDGF receptor status and compound I-response (FIG. 3) was striking. The analyses of PDGF receptor expression and phosphorylation indicated a strong co-variation between these parameters, indicating that ligand-production do not differ significantly between the cultures.

The absence of correlations between basal activation status of the down-stream signaling molecules Akt and ERK, and PDGF receptor status (FIG. 4) suggests that activation of Akt and ERK, in these GBM cultures, is under control of multiple signaling pathways. In this context it should be cautioned that both growth-inhibition experiments and analyses of Akt- and ERK-activation were performed in cells kept in the presence of 10% FCS. It is thus possible that potential influence of PDGF receptor signaling on these pathways, could be more clearly detected under other culture conditions.

The analysis on compound I-induced changes in Akt and ERK phosphorylation focused on ten selected GBM cultures which displayed a robust growth-inhibitory response (cultures 6, 7, 9 and 31) and six non-responders. Given the importance of Akt-signaling in PDGF receptor signaling it is somewhat surprising that growth inhibitory effects, presumably achieved through PDGF receptor inhibition, could be observed in the absence of consistent reduction in the activation of Akt (FIG. 5). One possible explanation for these findings is the presence of a PDGF receptor dependent pAkt, which is affected by PDGF receptor inhibition, but not detected in cells kept under culture conditions where serum components provide a high activation of Akt through PDGF independent pathways. It should also be cautioned that the cell lysates, analyzed for compound I-induced changes in signaling, were derived from cells that had only been exposed to compound I for 1 h. It should therefore be confirmed in a well-characterized PDGF-dependent control system that this length of time is sufficient to induce detectable changes in PDGF receptor-dependent phosphorylation of ERK and Akt.

The combined analyses of gene-expression profile and biochemical characterization revealed a series of interesting relationships (FIGS. 6 and 8). Briefly, cluster 1 is enriched in cultures displaying low PDGF receptor expression and low sensitivity to compound I, cluster 2 is enriched in compound I-responders with high PDGF receptor expression, and cluster 3 is composed mainly of cultures with a low growth rate with no consistent PDGF receptor expression. Preliminary analyses of the genes included in gene cluster III, IV and V (FIG. 7, Table 2) which are over-expressed in the GBM cluster 2 that is enriched in compound I-responders, have yet failed to suggest a particular developmental origin, or specific biological properties, of this GBM subset.

Using supervised analyses, based on the characterization of compound I-sensitivity and on the gene-expression analyses of this GBM panel, classifiers describing responders and non-responders were generated (FIG. 9, Table 3). Such classifiers can, in general, serve at least two purposes. Firstly, they can be used as starting points for development of diagnostic or prognostic tools. As long as performance of classifiers is good, this function of classifiers can be developed without any attention being paid to the biological significance of the genes making up the classifier. Secondly, classifiers can point towards the biological relationships which cause the two phenotypes, in this case compound I-sensitivity or -resistance, distinguished by the classifier.

### REFERENCES

Barbero, S., Bajetto, A., Bonavia, R., Porcile, C., Piccioli, P., Pirani, P., Ravetti, J. L., Zona, G., Spaziante, R., Florio, T., and Schettini, G. (2002). Expression of the chemokine receptor CXCR4 and its ligand stromal cell-derived factor 1 in human brain tumors and their involvement in glial proliferation in vitro. Ann. N.Y. Acad. Sci. 973, 60-69.
Capdeville, R., Buchdunger, E., Zimmermann, J., and Matter, A. (2002). Glivec (STI571, imatinib), a rationally developed, targeted anticancer drug. Nat. Rev. Drug Discov. 1, 493-502.
Claesson-Welsh, L., Eriksson, A., Morén, A., Severinsson, L., Ek, B., Östman, A., Betsholtz, C., and Heldin, C.-H. (1988). cDNA cloning and expression of a human platelet-derived growth factor (PDGF) receptor specific for B-chain-containing PDGF molecules. Mol. Cell. Biol. 8, 3476-3486.
Claesson-Welsh, L., Eriksson, A., Westermark, B., and Heldin, C.-H. (1989). cDNA cloning and expression of the human A-type platelet-derived growth factor (PDGF) receptor establishes structural similarity to the B-type PDGF receptor. P roc. Natl. Acad. Sci. USA 86, 4917-4921.
Dai, C., Celestino, J. C., Okada, Y., Louis, D. N., Fuller, G. N., and Holland, E. C. (2001). PDGF autocrine stimulation dedifferentiates cultured astrocytes and induces oligodendrogliomas and oligoastrocytomas from neural progenitors and astrocytes in vivo. Genes Dev. 15, 1913-1925.
Demetri, G. D., von Mehren, M., Blanke, C. D., Van den Abbeele, A. D., Eisenberg, B., Roberts, P. J., Heinrich, M. C., Tuveson, D. A., Singer, S., Janicek, M., Fletcher, J. A., Silverman, S. G., Silberman, S. L., Capdeville, R., Kiese, B., Peng, B., Dimitrijevic, S., Druker, B. J., Corless, C., Fletcher, C. D., and Joensuu, H. (2002). Efficacy and safety of imatinib mesylate in advanced gastrointestinal stromal tumors. N Engl J Med 347, 472-80.
Golub, T. R., Slonim, D. K., Tamayo, P., Huard, C., Gaasenbeek, M., Mesirov, J. P., Coller, H., Loh, M. L., Downing, J. R., Caligiuri, M. A., Bloomfield, C. D., and Lander, E. S. (1999). Molecular classification of cancer: class discovery and class prediction by gene expression monitoring. Science 286, 531-7.
Kilic, T., Alberta, J. A., Zdunek, P. R., Acar, M., Iannarelli, P., O'Reilly, T., Buchdunger, E., Black, P. M., and Stiles, C. D. (2000). Intracranial inhibition of platelet-derived growth factor-mediated glioblastoma cell growth by an orally active kinase inhibitor of the 2-phenylaminopyrimidine class. Cancer Res. 60, 5143-5150.
Lazarini, F., Tham, T. N., Casanova, P., Arenzana-Seisdedos, F., and Dubois-Dalcq, M. (2003). Role of the α-chemokine stromal cell-derived factor (SDF-1) in the developing and mature central nervous system. Glia 42, 139-148.
Maher, E A., Furnari, F B, Bachoo, R M, Rowitch, D H., Louis, D N., Cavanee, W C. and DePinho, R A. (2002) Malignant glioma: genetics and Biology of a grave matter. Genes and Dev. 15, 1311-1333
Mischel, P. S., Shai, R., Shi, T., Horvath, S., Lu, K. V., Choe, G., Seligson, D., Kremen, T. J., Palotie, A., Liau, L. M., Cloughesy, T. F., and Nelson, S. F. (2003). Identification of molecular subtypes of glioblastoma by gene expression profiling. Oncogene 22, 2361-73.
O'Brien, S. G., Guilhot, F., Larson, R. A., Gathmann, I., Baccarani, M., Cervantes, F., Cornelissen, J. J., Fischer, T., Hochhaus, A., Hughes, T., Lechner, K., Nielsen, J. L., Rousselot, P., Reiffers, J., Saglio, G., Shepherd, J., Simonsson, B., Gratwohl, A., Goldman, J. M., Kantarjian, H., Taylor, K., Verhoef, G., Bolton, A. E., Capdeville, R., and Druker, B. J. (2003). Imatinib compared with interferon and low-dose cytarabine for newly diagnosed chronic-phase chronic myeloid leukemia. N Engl J Med 348, 994-1004.
Pietras, K., Sjöblom, T., Rubin, K., Heldin, C.-H., and Östman, A. (2003). PDGF receptors as cancer drug targets. Cancer Cell 3, 439-443.
Ponten, J. and Westermark, B. (1987). Properties of human malignant glioma cells in vitro. Med. Biol. 56, 184-193
Rempel, S. A., Dudas, S., Ge, S., and Gutiérrez, J. A. (2000). Identification and localization of the cytokine SDF1 and its receptor, CXC chemokine receptor 4, to regions of necrosis and angiogenesis in human glioblastoma. Clin. Cancer Res. 6, 102-111.
Rubin, J. B., Kung, A. L., Klein, R. S., Chan, J. A., Sun, Y., Schmidt, K., Kieran, M. W., Luster, A. D., and Segal, R. A. (2003). A small-molecule antagonist of CXCR4 inhibits intracranial growth of primary brain tumors. Proc. Natl. Acad. Sci. USA 100, 13513-13518.
Shamah, S. M., Stiles, C. D., and Guha, A. (1993). Dominant-negative mutants of platelet-derived growth factor revert the transformed phenotype of human astrocytoma cells. Mol. Cell. Biol. 13, 7203-7212.
Sjöblom, T., Shimizu, A., O'Brien, K. P., Pietras, K., Dal Cin, P., Buchdunger, E., Dumanski, J. P., Östman, A., and Heldin, C.-H. (2001). Growth inhibition of dermatofibrosarcoma protuberans tumors by the platelet-derived growth factor receptor antagonist STI571 through induction of apoptosis. Cancer Res. 61, 5778-5783.
Strawn, L. M., Mann, E., Elliger, S. S., Chu, L. M., Germain, L. L., Niederfellner, G., Ullrich, A., and Shawver, L. K. (1994). Inhibition of glioma cell growth by a truncated platelet-derived growth factor-β receptor. J. Biol. Chem. 269, 21215-21222.
Tamayo, P., Slonim, D., Mesirov, J., Zhu, Q., Kitareewan, S., Dmitrovsky, E., Lander, E. S., and Golub, T. R. (1999). Interpreting patterns of gene expression with self-organizing maps: methods and application to hematopoietic differentiation. Proc Natl Acad Sci USA 96, 2907-12.
Uhrbom, L., Hesselager, G., Nistér, M., and Westermark, B. (1998). Induction of brain tumors in mice using a recombinant platelet-derived growth factor B-chain retrovirus. Cancer Res. 58, 5275-5279.
Van Gelder, R. N., von Zastrow, M. E., Yool, A., Dement, W. C., Barchas, J. D., and Eberwine, J. H. (1990). Amplified RNA synthesized from limited quantities of heterogeneous cDNA. Proc. Natl. Acad. Sci. USA 87, 1663-1667.

TABLE 1

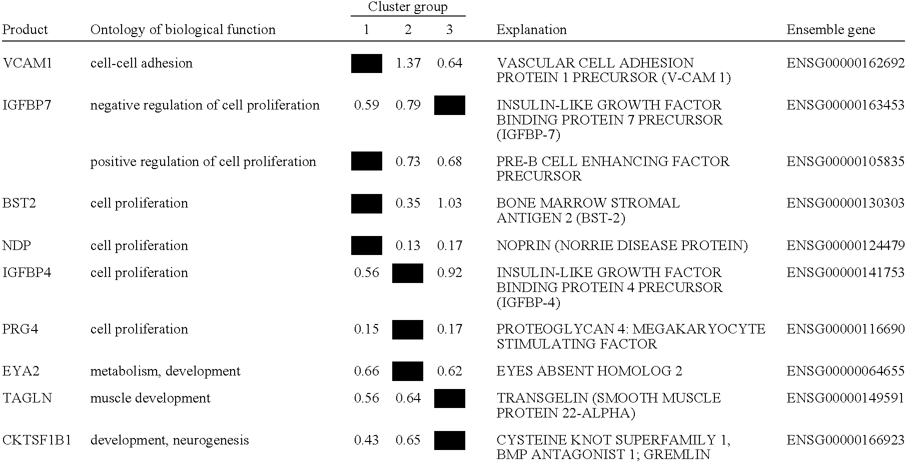

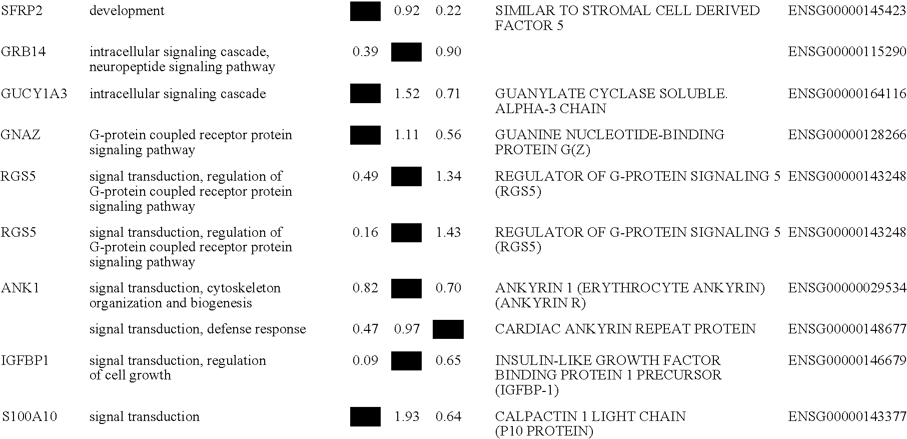

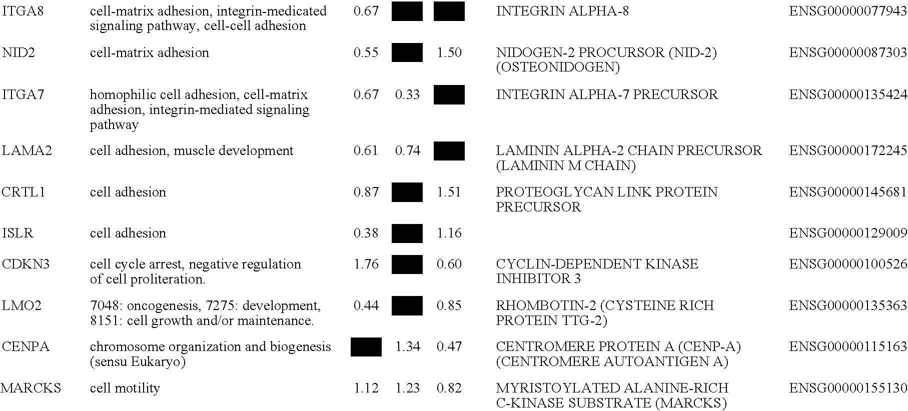

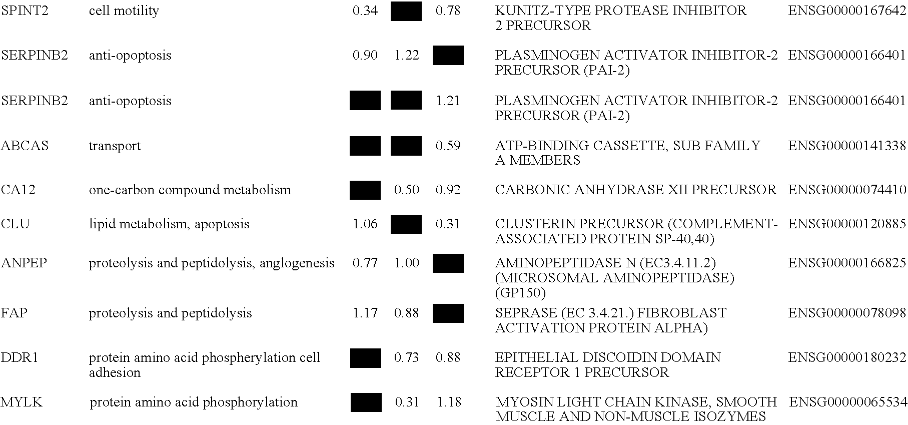

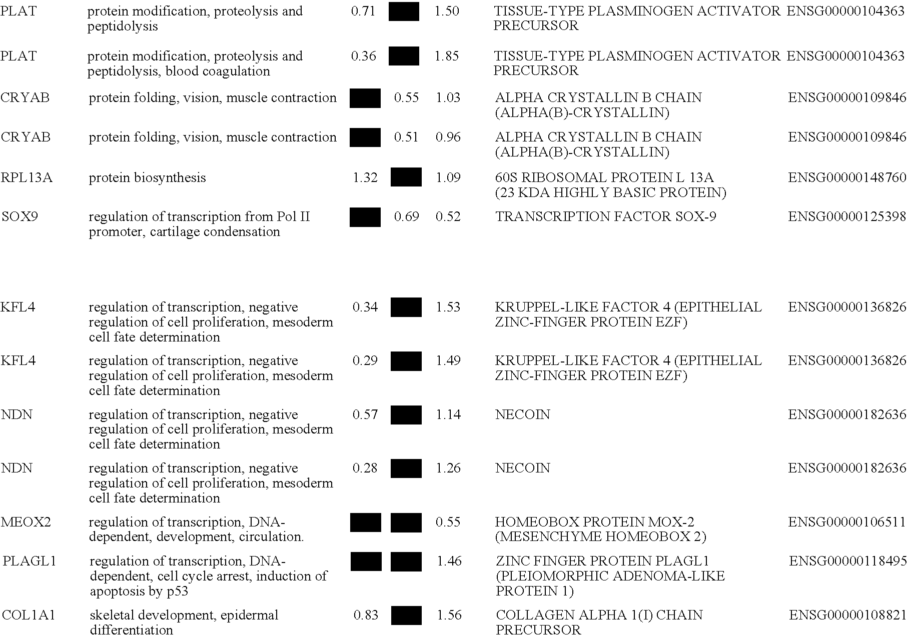

List of differentially expressed genes from the feature list used to generate the GBM clustering shown in FIG. 6A and FIG. 7. Genes are sorted according to function as defined by gene ontology.

TABLE 2

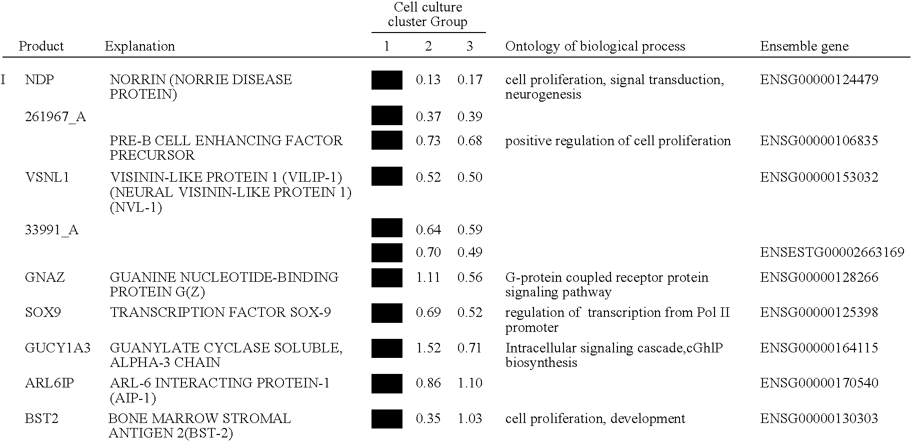

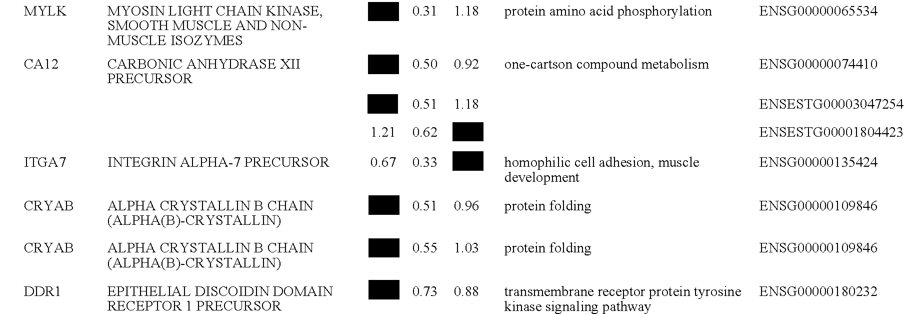

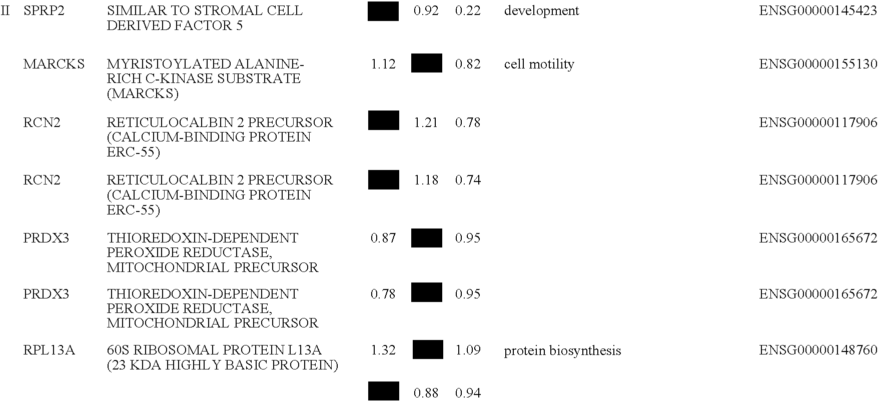

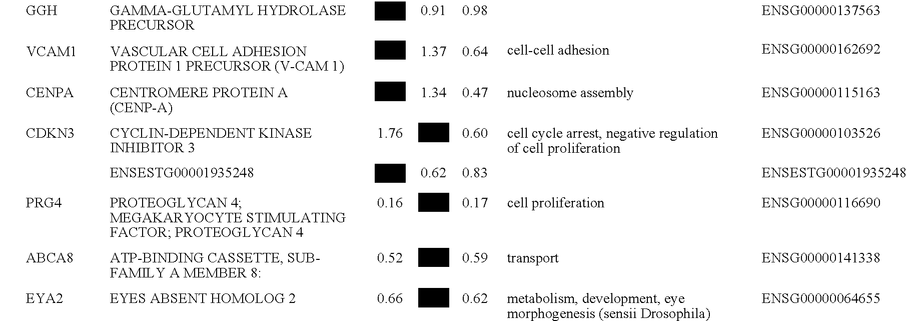

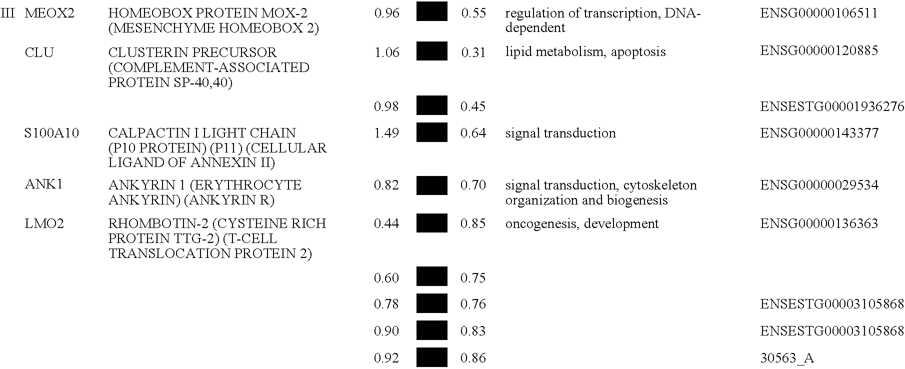

List of differentially expressed genes from the feature list used to generate the GBM clustering shown in FIG. 6A and FIG. 7. Genes are sorted according to the results from the clustering analyses of the differentially expressed genes (FIG. 7).

TABLE 3

| | | Cell culture | | | | | | | | | | |
|---|---|---|---|---|---|---|---|---|---|---|---|---|
| | | 31 | 9 | 7 | 6 | 5 | 38 | 18 | 35 | 21 | 30 | |
| | times | Class | | | | | | | | | | |
| common | used | R | R | R | R | NR | NR | NR | NR | NR | NR | Description |

genes used for a 4 feature list in leave one cut test

| | | | | | | | | | | | | |
|---|---|---|---|---|---|---|---|---|---|---|---|---|
| CXCL12 | 10 | 2.0 | 1.7 | 2.0 | 1.5 | 0.4 | 0.3 | 0.1 | 0.4 | 0.1 | 0.2 | STROMAL CELL-DERIVED FACTOR 1 PRECURSOR (SDF-1) (CXCL12) |
| CXCL12 | 10 | 1.8 | 1.7 | 1.9 | 1.5 | 0.4 | 0.6 | 0.2 | 0.5 | 0.1 | 0.1 | STROMAL CELL-DERIVED FACTOR 1 PRECURSOR (SDF-1) (CXCL12) |
| CKTSF1B1 | 5 | 1.3 | 1.4 | 1.6 | 0.4 | 0.1 | 0.3 | 0.1 | 0.1 | 0.0 | 0.0 | CYSTEINE KNOT SUPERFAMILY 1, BMP ANTAGONIST 1; GREMLIN |
| COL1A1 | 4 | 1.9 | 1.7 | 1.7 | 2.3 | 1.1 | 0.6 | 0.2 | 0.4 | 0.3 | 0.5 | COLLAGEN ALPHA 1(I) CHAIN PRECURSOR |
| COL1A1 | 4 | 1.5 | 1.3 | 1.4 | 1.8 | 0.7 | 0.6 | 0.2 | 0.3 | 0.1 | 0.4 | COLLAGEN ALPHA 1(I) CHAIN PRECURSOR |
| ITGA8 | 2 | 1.6 | 3.2 | 2.7 | 1.7 | 0.5 | 0.5 | 0.7 | 0.3 | 0.5 | 0.3 | INTEGRIN ALPHA-8 |
| SLC6A8 | 1 | 0.4 | 0.3 | 0.4 | 0.4 | 0.5 | 1.4 | 1.3 | 2.6 | 0.8 | 1.3 | SODIUM- AND CHLORIDE-DEPENDENT CREATINE TRANSPORTER 1 |
| | | | | | | | | | | | | (CT1) |
| COX7B | 1 | 0.5 | 0.4 | 0.6 | 0.5 | 1.1 | 1.3 | 1.4 | 1.3 | 0.9 | 0.5 | CYTOCHROMEC OXIDASE POLYPEPTIDE VIIB |
| | 1 | 1.0 | 0.8 | 1.2 | 1.2 | 0.5 | 0.7 | 0.3 | 0.2 | 0.3 | 0.4 | ENSESTG00002766964 |
| NDN | 1 | 0.7 | 2.3 | 1.4 | 1.8 | 0.6 | 0.6 | 0.4 | 0.2 | 0.1 | 0.3 | NECDIN |
| COL1A1 | 1 | 1.8 | 1.8 | 1.8 | 2.3 | 1.1 | 0.6 | 0.2 | 0.4 | 0.4 | 0.0 | COLLAGEN ALPHA 1(I) CHAIN PRECURSOR |
| sum | 40 | | | | | | | | | | | |

genes used for a 10 feature list in leave one cut test

| | | | | | | | | | | | | |
|---|---|---|---|---|---|---|---|---|---|---|---|---|
| CXCL12 | 10 | 2.0 | 1.7 | 2.0 | 1.5 | 0.4 | 0.3 | 0.1 | 0.4 | 0.1 | 0.2 | STROMAL CELL-DERIVED FACTOR 1 PRECURSOR (SDF-1) (CXCL12) |
| COL1A1 | 10 | 1.9 | 1.7 | 1.7 | 2.3 | 1.1 | 0.6 | 0.2 | 0.4 | 0.3 | 0.5 | COLLAGEN ALPHA 1(I) CHAIN PRECURSOR |
| CXCL12 | 10 | 1.8 | 1.7 | 1.9 | 1.5 | 0.4 | 0.6 | 0.2 | 0.5 | 0.1 | 0.1 | STROMAL CELL-DERIVED FACTOR 1 PRECURSOR (SDF-1) (CXCL12) |
| CDL1A1 | 10 | 1.5 | 1.3 | 1.4 | 1.8 | 0.7 | 0.6 | 0.2 | 0.3 | 0.1 | 0.4 | COLLAGEN ALPHA 1(I) CHAIN PRECURSOR |
| COL1A1 | 9 | 1.8 | 1.8 | 1.8 | 2.3 | 1.1 | 0.6 | 0.2 | 0.4 | 0.4 | 0.6 | COLLAGEN ALPHA 1(I) CHAIN PRECURSOR |
| AEBP1 | 8 | 1.6 | 1.8 | 1.7 | 1.2 | 1.0 | 0.6 | 0.8 | 0.5 | 0.4 | 0.4 | ADIPOCYTE ENHANCER BINDING PROTEIN 1 PRECURSOR; |
| | | | | | | | | | | | | AE-BINDING PROTEIN 1 |
| CKTSF1B1 | 8 | 1.3 | 1.4 | 1.5 | 0.4 | 0.1 | 0.3 | 0.1 | 0.1 | 0.0 | 0.0 | CYSTEINE KNOT SUPERFAMILY 1, BMP ANTAGONIST 1; GREMLIN |
| AEBP1 | 7 | 1.6 | 1.7 | 1.6 | 1.2 | 0.9 | 0.7 | 0.8 | 0.6 | 0.4 | 0.5 | ADIPOCYTE ENHANCER BINDING PROTEIN 1 PRECURSOR; |
| | | | | | | | | | | | | AE-BINDING PROTEIN 1 |
| PTGFR | 6 | 1.3 | 1.4 | 1.3 | 0.9 | 0.7 | 0.8 | 0.6 | 0.5 | 0.5 | 0.6 | PROSTAGLANDIN F2-ALPHA RECEPTOR |
| | 6 | 1.0 | 0.8 | 1.2 | 1.2 | 0.5 | 0.7 | 0.3 | 0.2 | 0.3 | 0.4 | ENSESTG00002766964 |
| ITGA8 | 5 | 1.6 | 3.2 | 2.7 | 1.7 | 0.5 | 0.5 | 0.7 | 0.3 | 0.5 | 0.3 | INTEGRIN ALPHA-8 |
| FOXF2 | 2 | 1.6 | 1.7 | 2.6 | 0.7 | 0.2 | 0.7 | 0.2 | 0.2 | 0.4 | 0.2 | FORKHEAD BOX PROTEIN F2 (FORKHEAD-RELATED PROTEIN FKHL6) |
| COX7B | 1 | 0.5 | 0.4 | 0.6 | 0.5 | 1.1 | 1.3 | 1.4 | 1.3 | 0.9 | 0.5 | CYTOCHROME C OXIDASE POLYPEPTIDE VIIB |
| SLC6A8 | 1 | 0.4 | 0.3 | 0.4 | 0.4 | 0.5 | 1.4 | 1.3 | 2.6 | 0.8 | 1.3 | SODIUM- AND CHLORIDE-DEPENDENT CREATINE TRANSPORTER 1 |
| | | | | | | | | | | | | (CT1) |
| | 1 | 1.1 | 0.8 | 1.3 | 1.8 | 0.5 | 0.7 | 0.4 | 0.4 | 0.3 | 0.4 | ENSESTG00002766964 |
| | 1 | 1.7 | 2.3 | 1.7 | 1.2 | 1.7 | 0.5 | 0.2 | 0.1 | 0.4 | 0.6 | ENSG00000170714 |
| NDN | 1 | 0.7 | 2.3 | 1.4 | 1.8 | 0.6 | 0.6 | 0.4 | 0.2 | 0.1 | 0.3 | NECDIN |
| | 1 | 1.5 | 1.0 | 1.4 | 1.8 | 1.7 | 0.6 | 0.3 | 0.2 | 0.4 | 0.7 | |
| FAP | 1 | 1.4 | 1.7 | 1.3 | 0.7 | 0.4 | 0.7 | 0.4 | 0.6 | 0.3 | 0.7 | SEPRASE, FIBROBLAST ACTIVATION PROTEIN ALPHA |
| VSNL1 | 1 | 0.5 | 0.4 | 0.6 | 0.4 | 0.6 | 1.4 | 2.2 | 2.4 | 2.4 | 0.3 | VISININ-LIKE PROTEIN 1 (VILIP-1) (NEURAL VISININ-LIKE PROTEIN 1) |
| PHYH | 1 | 0.5 | 0.5 | 0.7 | 0.8 | 1.1 | 1.1 | 1.4 | 1.5 | 1.4 | 0.8 | PHYTANOYL-COA DIOXY GENASE, PEROXISOMAL PRECURSOR |
| sum | 100 | | | | | | | | | | | |

List of the features included in the 4-feature (upper part) and 10-feature (lower part)
classifiers used in the weighted-voting classification of the 10 GBM cultures.
Relative expression of these features in the 10 GBM cultures included in the analyses is also shown.

TABLE 4

| | | | | Training set | | | | | | | | | | | | | | | | |
|---|---|---|---|---|---|---|---|---|---|---|---|---|---|---|---|---|---|---|---|---|
| | | | | cell lines | | | | | | | | | | | decission | Test set | | | | |
| nr of features | | | common | 31 | 9 | 7 | 6 | 5 | 18 | 38 | 35 | 21 | 30 | signal to noise | boundary | 11 | 45 | 8 | 27 | 34 |
| 5 | 4 | 3 | CXCL12 | 2.0 | 1.7 | 2.0 | 1.5 | 0.4 | 0.1 | 0.3 | 0.4 | 0.1 | 0.2 | 4.2 | 0.76 | 1.9 | 0.9 | 0.7 | 1.2 | 0.6 |
| | | | CXCL12 | 1.8 | 1.7 | 1.9 | 1.5 | 0.4 | 0.2 | 0.6 | 0.5 | 0.1 | 0.1 | 3.7 | 0.71 | 1.7 | 0.9 | 0.8 | 1.1 | 0.8 |
| | | | COL1A1 | 1.8 | 1.8 | 1.8 | 2.3 | 1.1 | 0.2 | 0.6 | 0.4 | 0.4 | 0.6 | 2.4 | 0.69 | 2.3 | 2 | 1.6 | 1.7 | 1.7 |
| | | | COL1A1 | 1.5 | 1.3 | 1.4 | 1.8 | 0.7 | 0.2 | 0.6 | 0.3 | 0.1 | 0.4 | 2.3 | 0.56 | 1.9 | 1.5 | 1.2 | 1.5 | 1.3 |
| | | | COL1A1 | 1.9 | 1.7 | 1.7 | 2.3 | 1.1 | 0.2 | 0.6 | 0.4 | 0.3 | 0.5 | 2.3 | 0.72 | 2.2 | 1.9 | 1.5 | 1.8 | 1.6 |

| | | | | | | | | | | | | | | | |
|---|---|---|---|---|---|---|---|---|---|---|---|---|---|---|---|
| empiricaly determined class | R | R | R | R | NR | NR | NR | NR | NR | NR | R | R | I | I | NR |
| class according to classifier | | | | | | | | | | | | | | | |
| 3 features | | | | | | | | | | | R | R | NR | R | NR |
| 4 features | | | | | | | | | | | R | R | NR | R | NR |
| 5 features | | | | | | | | | | | R | R | NR | R | NR |

Composition of classifiers, with 3, 4 or 5 features, generated from the top features in a signal to noise ranked gene list from 10 glioblastoma cell cultures. Relative expression of these features in the 10 GBM cultures of the training-set, and the 5 GBM culltures of the test set are indicated. Signal to noise is based on differences between mean expression in the two groups Decision boundary is the average of the the expression averages in the responders and non-responders.

1. A method for in vitro diagnosing a cell proliferative disease in a mammal, comprising at least:
a) providing a biological sample from said mammal;
b) determining the expression and/or phosphorylation profile in said sample of at least 2 to 40 genetic markers selected from Table 3,
c) comparing patients gene expression profile to the mean Non-responsive expression profiles shown in Tables 3;
d) determining the similarity between the two gene expression profiles resulting from the comparison in (b);
e) determining the likelihood that the patient has a GBM condition responsive to a drug by means of the degree of similarity determined in (c).

2. The method according to claim 1, wherein the expression and/or phosphorylation profile of at least 3 to 5 genetic markers selected from Table 3 is determined in step b).

3. A method for predicting the behaviour of a mammal having a cell proliferative disease, in response to a medical treatment using at least one PDGF receptor antagonist, comprising at least:
a) providing a biological sample from said mammal;
b) determining the expression and/or phosphorylation profile in said sample of at least 2 to 40 genetic markers selected from Table 3;
c) comparing the expression and/or phosphorylation profile obtained in step b) to the means ±standard deviations calculated from Table 3 for responsive and non-responsive expression and/or phosphorylation profiles; and
d) predicting the behaviour of said mammal as follows:
when the expression and/or phosphorylation profile obtained in b) is in the mean ±standard deviation calculated for responsive expression and/or phosphorylation profiles, then said mammal is predicted to be responsive to said treatment;
when the expression and/or phosphorylation profile obtained in b) is in the mean ±standard deviation calculated for non-responsive expression and/or phosphorylation profiles, then said mammal is predicted to be non-responsive to said treatment; and
when the expression and/or phosphorylation profile obtained in b) is out of the means ±standard deviations calculated for responsive and non-responsive expression and/or phosphorylation profiles, then the behaviour of said mammal in response to said treatment is undetermined.

4. The method according to claim 3, wherein the expression and/or phosphorylation profile of at least 3 to 5 genetic markers selected from Table 3 is determined in step b).

5. A method for selecting a mammal having a cell proliferative disease, wherein said mammal is predicted to be responsive to a medical treatment using at least one PDGF receptor antagonist, comprising at least:
a) predicting the behaviour of said mammal by using the method according to claim 3; and
b) if said mammal is predicted to be responsive, then selecting said mammal.

6. The method according to claim 1, wherein said mammal is a human.

7. A kit for in vitro analyzing the expression and/or phosphorylation profile of genetic markers in a mammal, comprising cDNAs and/or antibodies for at least 2 to 40 genetic markers selected from Table 3.

8. The kit according to claim 7, comprising cDNAs and/or antibodies for at least 3 to 5 genetic markers selected from Table 3.

9. The kit according to claim 7, wherein said mammal is a human.

10. A microarray or a biochip for in vitro analyzing the expression and/or phosphorylation profile of genetic markers in a mammal, comprising cDNAs and/or antibodies for at least 2 to 40 genetic markers selected from Table 3.

11. The microarray or the biochip according to claim 10, comprising cDNAs and/or antibodies for at least 3 to 5 genetic markers selected from Table 3.

12. The microarray or the biochip according to claim 10, wherein said mammal is a human.

13. Use of at least one gene and/or at least one gene product selected from Table 3 as a genetic marker for:
in vitro diagnosing a cell proliferative disease in a mammal; and/or
predicting the behaviour of a mammal having a cell proliferative disease in response to a medical treatment using at least one PDGF receptor antagonist; and/or
selecting a mammal having a cell proliferative disease, wherein said mammal is predicted to be responsive to a medical treatment using at least one PDGF receptor antagonist.

14. The use according to claim 13, wherein cDNA corresponding to said gene and/or antibody(ies) specific for said gene product is used.

15. Use of the kit according to claim 7 for:
in vitro diagnosing a cell proliferative disease in a mammal; and/or
predicting the behaviour of a mammal having a cell proliferative disease in response to a medical treatment using at least one PDGF receptor antagonist; and/or
selecting a mammal having a cell proliferative disease, wherein said mammal is predicted to be responsive to a medical treatment using at least one PDGF receptor antagonist.

16. Use of the microarray or the biochip according to claim 10 for:
in vitro diagnosing a cell proliferative disease in a mammal; and/or
predicting the behaviour of a mammal having a cell proliferative disease in response to a medical treatment using at least one PDGF receptor antagonist; and/or
selecting a mammal having a cell proliferative disease, wherein said mammal is predicted to be responsive to a medical treatment using at least one PDGF receptor antagonist.

17. Use of at least one PDGF receptor antagonist for the manufacture of a drug for treating a responsive mammal having a cell proliferative disease, wherein said responsive mammal is selected using the method according to claim 5.

18. The use according to claim 13, wherein said mammal is a human.
